(19) United States
(12) Patent Application Publication
(10) Pub. No.: US 2011/0009482 A1
(43) Pub. Date: Jan. 13, 2011

(54) METHODS OF TREATING COPD

(75) Inventors: Irina V. Khanskaya, Morristown, NJ (US); Jonathan Sadeh, New York, NY (US); Heribeth Staudinger, Green Brook, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation, Kenilworth, NJ (US)

(21) Appl. No.: 12/746,232

(22) PCT Filed: Dec. 3, 2008
(86) PCT No.: PCT/US08/85327

§ 371 Date: Sep. 15, 2010

Related U.S. Application Data
(60) Provisional application No. 60/992,190, filed on Dec. 4, 2007.

(51) Int. Cl. A61K 31/366; A61K 31/341; A61P 11/00

(52) U.S. Cl. 514/460; 514/471

(57) ABSTRACT

Disclosed is a method of treating chronic obstructive disease, said method comprising administering an effective amount of a CXCR2 antagonist and administering an effective amount of at least one drug selected from the group consisting of: angiotensin-converting enzyme inhibitors, Angiotensin II receptor antagonists, cardioselective beta blockers, and lipid regulating drugs. Examples of the CXCR2 antagonist include: (formula 1.0A and 1.0B).

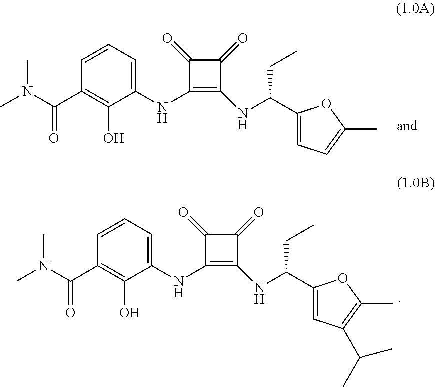

### REFERENCE TO RELATED APPLICATION

This application claims the benefit of U.S. Provisional Application Ser. No. 60/992,190 filed Dec. 4, 2007.

### BACKGROUND

In view of the present interest in treating Chronic Obstructive Pulmonary Disease (COPD), and in alleviating the symptoms of COPD, a method for treating COPD would be a welcome contribution to the art. This invention provides such a contribution.

### SUMMARY OF THE INVENTION

This invention provides a method of treating chronic obstructive disease (COPD) in a patient in need of such treatment. The method comprises administering to the patient an effective amount of at least one (e.g., 1, 2 or 3, or 1 or 2, or 1, and usually 1) CXCR2 antagonist, and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a method of treating chronic obstructive disease in a patient in need of such treatment wherein said method comprises administering to the patient an effective amount a CXCR2 antagonist, and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention provides a method of treating chronic obstructive disease in a patient in need of such treatment wherein said method comprises administering to the patient an effective amount of a pharmaceutical composition comprising at least one (e.g., 1, 2 or 3, or 1 or 2, or 1, and usually 1) CXCR2 antagonist and a pharmaceutically acceptable carrier, and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a method of treating chronic obstructive disease in a patient in need of such treatment wherein said method comprises administering to the patient an effective amount a pharmaceutical composition comprising a CXCR2 antagonist and a pharmaceutically acceptable carrier, and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a pharmaceutical composition comprising an effective amount of at least one (e.g., 1, 2 or 3, or 1 or 2, or 1, and usually 1) CXCR2 antagonist, a pharmaceutically acceptable carrier, and an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a pharmaceutical composition comprising an effective amount of a CXCR2 antagonist, a pharmaceutically acceptable carrier, and an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a pharmaceutical composition comprising an effective amount of a CXCR2 antagonist, a pharmaceutically acceptable carrier, and an effective amount of a lipid regulating drug (i.e., a statin).

This invention also provides a method of treating chronic obstructive disease in a patient in need of such treatment wherein said method comprises administering to the patient an effective amount a pharmaceutical composition, said pharmaceutical composition comprises an effective amount of at least one (e.g., 1, 2 or 3, or 1 or 2, or 1, and usually 1) CXCR2 antagonist, and an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a method of treating chronic obstructive disease in a patient in need of such treatment wherein said method comprises administering to the patient an effective amount a pharmaceutical composition, said pharmaceutical composition comprises an effective amount a CXCR2 antagonist, and an effective amount of a drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

This invention also provides a method of treating chronic obstructive disease in a patient in need of such treatment wherein said method comprises administering to the patient an effective amount a pharmaceutical composition, said pharmaceutical composition comprises an effective amount a CXCR2 antagonist, and an effective amount of a lipid regulating drug (statin).

This invention also provides a method of treating chronic obstructive disease in a patient in need of such treatment wherein said method comprises administering to the patient an effective amount a pharmaceutical composition, said pharmaceutical composition comprises an effective amount a CXCR2 antagonist, and an effective amount of Simvastatin.

### DETAILED DESCRIPTION OF THE INVENTION

Examples of CXCR2 antagonists include those described in U.S. Pat. No. 7,132,445 issued on Nov. 7, 2006, and WO 02/083624 published Oct. 24, 2002, the disclosures of each being incorporated herein by reference thereto.

In one embodiment of this invention the CXCR2 antagonist used is a compound of the formula:

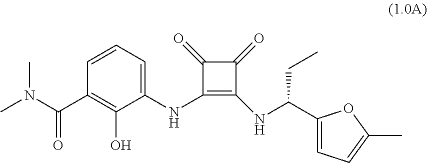

or a pharmaceutically acceptable salt, solvate, ester, or polymorph thereof.

The compound of formula (1.0A) is described in U.S. Pat. No. 7,132,445.

In another embodiment of this invention the CXCR2 antagonist used is a compound of the formula:

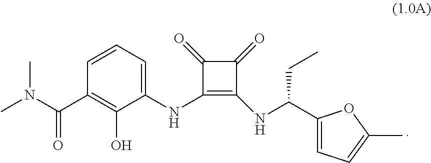

In another embodiment of this invention the CXCR2 antagonist used is a solvate of the compound of the formula (1.0A).

In another embodiment of this invention the CXCR2 antagonist used is a monohydrate of the compound of the formula (1.0A).

In another embodiment of this invention the CXCR2 antagonist used is a pharmaceutically acceptable salt of the compound of the formula (1.0A).

In another embodiment of this invention the CXCR2 antagonist used is a polymorph of the compound of the formula (1.0A).

In another embodiment of this invention the CXCR2 antagonist used is a polymorph of the compound of the formula (1.0A), and said polymorph is Form I.

In another embodiment of this invention the CXCR2 antagonist used is a polymorph of the compound of the formula (1.0A), and said polymorph is Form II.

In another embodiment of this invention the CXCR2 antagonist used is a polymorph of the compound of the formula (1.0A), and said polymorph is Form III.

In another embodiment of this invention the CXCR2 antagonist used is a polymorph of the compound of the formula (1.0A), and said polymorph is Form IV.

Polymorph Forms I, II, III, and IV of formula (1.0A) are identified in WO2005/075447 published Aug. 18, 2005, as well as in the counterpart U.S.2005/0192345 published Sep. 1, 2005, the disclosures of each being incorporated herein by reference thereto. Formulations of formula (1.0A) are described in US2008/002110 published Jan. 24, 2008) and WO2007/146296 (published Dec. 21, 2007).

In another embodiment of this invention the CXCR2 antagonist used is a compound of the formula:

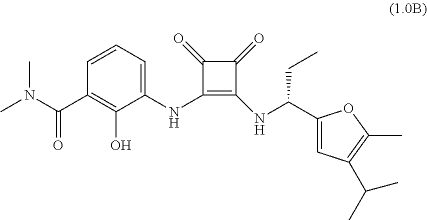

or a pharmaceutically acceptable, salt, solvate, ester or polymorph thereof.

Compounds of formula (1.0B) are described in PCT Application No. US2007/015671 filed on Jul. 5, 2007 (published as WO2008/005570 on Jan. 10, 2008), and U.S. application Ser. No. 11/773,479 filed on Jul. 5, 2007 (published as US2008/0045489 on Feb. 21, 2008), the disclosures of which are incorporated herein by reference thereto.

In another embodiment of this invention the CXCR2 antagonist used is a compound of the formula (1.0B).

In another embodiment of this invention the CXCR2 antagonist used is a solvate of the compound of the formula (1.0B).

In another embodiment of this invention the CXCR2 antagonist used is a monohydrate of the compound of the formula (1.0B).

In another embodiment of this invention the CXCR2 antagonist used is a pharmaceutically acceptable salt of the compound of the formula (1.0B).

In another embodiment of this invention the CXCR2 antagonist used is a polymorph of the compound of the formula (1.0B).

In another embodiment of this invention the compound of formula (1.0A) is used in amounts of about 3 mg to about 30 mg administered daily.

In another embodiment of this invention about 3 mg of the compound of formula (1.0A) is administered daily.

In another embodiment of this invention about 10 mg of the compound of formula (1.0A) is administered daily.

In another embodiment of this invention about 30 mg of the compound of formula (1.0A) is administered daily.

In another embodiment of this invention a solvate of the compound of formula (1.0A) is used in amounts of about 3 mg to about 30 mg administered daily.

In another embodiment of this invention about 3 mg of a solvate of the compound of formula (1.0A) is administered daily.

In another embodiment of this invention about 10 mg of a solvate of the compound of formula (1.0A) is administered daily.

In another embodiment of this invention about 30 mg of a solvate of the compound of formula (1.0A) is administered daily.

in another embodiment of this invention a monohydrate of the compound of formula (1.0A) is used in amounts of about 3 mg to about 30 mg administered daily.

In another embodiment of this invention about 3 mg of a monohydrate of the compound of formula (1.0A) is administered daily.

In another embodiment of this invention about 10 mg of a monohydrate of the compound of formula (1.0A) is administered daily.

In another embodiment of this invention about 30 mg of a monohydrate of the compound of formula (1.0A) is administered daily.

In another embodiment of this invention a pharmaceutically acceptable salt of the compound of formula (1.0A) is used in amounts of about 3 mg to about 30 mg administered daily.

In another embodiment of this invention about 3 mg of a pharmaceutically acceptable salt of the compound of formula (1.0A) is administered daily.

In another embodiment of this invention about 10 mg of a pharmaceutically acceptable salt of the compound of formula (1.0A) is administered daily.

In another embodiment of this invention about 30 mg of a pharmaceutically acceptable salt of the compound of formula (1.0A) is administered daily.

In another embodiment of this invention a pharmaceutically acceptable ester of the compound of formula (1.0A) is used in amounts of about 3 mg to about 30 mg administered daily.

In another embodiment of this invention about 3 mg of a pharmaceutically acceptable ester of the compound of formula (1.0A) is administered daily.

In another embodiment of this invention about 10 mg of a pharmaceutically acceptable ester of the compound of formula (1.0A) is administered daily.

In another embodiment of this invention about 30 mg of a pharmaceutically acceptable ester of the compound of formula (1.0A) is administered daily,

In another embodiment of this invention the Form I polymorph of the compound of formula (1.0A) is used in amounts of about 3 mg to about 30 mg administered daily.

In another embodiment of this invention about 3 mg of the Form I polymorph of the compound of formula (1.0A) is administered daily.

In another embodiment of this invention about 10 mg of the Form I polymorph of the compound of formula (1.0A) is administered daily.

In another embodiment of this invention about 30 mg of the Form I polymorph of the compound of formula (1.0A) is administered daily.

In another embodiment of this invention the Form II polymorph of the compound of formula (1.0A) is used in amounts of about 3 mg to about 30 mg administered daily.

In another embodiment of this invention about 3 mg of the Form II polymorph of the compound of formula (1.0A) is administered daily.

In another embodiment of this invention about 10 mg of the Form II polymorph of the compound of formula (1.0A) is administered daily.

In another embodiment of this invention about 30 mg of the Form II polymorph of the compound of formula (1.0A) is administered daily.

In another embodiment of this invention the Form III polymorph of the compound of formula (1.0A) is used in amounts of about 3 mg to about 30 mg administered daily.

In another embodiment of this invention about 3 mg of the Form III polymorph of the compound of formula (1.0A) is administered daily.

In another embodiment of this invention about 10 mg of the Form III polymorph of the compound of formula (1.0A) is administered daily.

In another embodiment of this invention about 30 mg of the Form III polymorph of the compound of formula (1.0A) is administered daily.

In another embodiment of this invention the Form IV polymorph of the compound of formula (1.0A) is used in amounts of about 3 mg to about 30 mg administered daily.

In another embodiment of this invention about 3 mg of the Form IV polymorph of the compound of formula (1.0A) is administered daily.

In another embodiment of this invention about 10 mg of the Form IV polymorph of the compound of formula (1.0A) is administered daily.

In another embodiment of this invention about 30 mg of the Form IV polymorph of the compound of formula (1.0A) is administered daily.

In another embodiment of this invention the compound of formula (1.0B) is used in amounts of about 3 mg to about 30 mg administered daily.

In another embodiment of this invention about 3 mg of the compound of formula (1.0B) is administered daily.

In another embodiment of this invention about 10 mg of the compound of formula (1.0B) is administered daily.

In another embodiment of this invention about 30 mg of the compound of formula (1.0B) is administered daily.

In another embodiment of this invention a solvate of the compound of formula (1.0B) is used in amounts of about 3 mg to about 30 mg administered daily.

In another embodiment of this invention about 3 mg of a solvate of the compound of formula (1.0B) is administered daily.

In another embodiment of this invention about 10 mg of a solvate of the compound of formula (1.0B) is administered daily.

In another embodiment of this invention about 30 mg of a solvate of the compound of formula (1.0B) is administered daily.

In another embodiment of this invention a monohydrate of the compound of formula (1.0B) is used in amounts of about 3 mg to about 30 mg administered daily.

In another embodiment of this invention about 3 mg of a monohydrate of the compound of formula (1.0B) is administered daily.

In another embodiment of this invention about 10 mg of a monohydrate of the compound of formula (1.0B) is administered daily.

In another embodiment of this invention about 30 mg of a monohydrate of the compound of formula (1.0B) is administered daily.

In another embodiment of this invention a pharmaceutically acceptable salt of the compound of formula (1.0B) is used in amounts of about 3 mg to about 30 mg administered daily.

In another embodiment of this invention about 3 mg of a pharmaceutically acceptable salt of the compound of formula (1.0B) is administered daily.

In another embodiment of this invention about 10 mg of a pharmaceutically acceptable salt of the compound of formula (1.0B) is administered daily.

In another embodiment of this invention about 30 mg of a pharmaceutically acceptable salt of the compound of formula (1.0B) is administered daily.

In another embodiment of this invention a pharmaceutically acceptable ester of the compound of formula (1.0B) is used in amounts of about 3 mg to about 30 mg administered daily.

In another embodiment of this invention about 3 mg of a pharmaceutically acceptable ester of the compound of formula (1.0B) is administered daily.

In another embodiment of this invention about 10 mg of a pharmaceutically acceptable ester of the compound of formula (1.0B) is administered daily.

In another embodiment of this invention about 30 mg of a pharmaceutically acceptable ester of the compound of formula (1.0B) is administered daily.

In another embodiment of this invention about 3 mg of a polymorph of the compound of formula (1.0B) is administered daily.

In another embodiment of this invention about 10 mg of a polymorph of the compound of formula (1.0B) is administered daily.

In another embodiment of this invention about 30 mg of a polymorph of the compound of formula (1.0B) is administered daily.

The dosages of the compound of formula (1.0A) or (1.0B), in the embodiments above, can be given as a single dose, or can be given in divided doses (e.g., two divided doses).

The angiotensin-converting enzyme (ACE) inhibitors, Angiotensin II receptor antagonists (Angiotensin II receptor blockers), cardioselective beta blockers, and lipid regulating drugs (statins) can be administered according to known protocols, such as, for example, the protocols described in the Physicians Desk Reference (see for example, the Physicians' Desk Reference, 2006, published by Thompson PDR at Montvale, N.J. 07645-1742, the disclosure of which is incorporated herein by reference thereto).

Examples of said angiotensin-converting enzyme (ACE) inhibitors include, but are not limited to: (a) Benazepril HCl, (b) Captopril, (c) Moexipril hydrochloride, (d) Perindopril erbumine, (e) Lisinopril, (f) Ramipril, and (g) Trandolapril.

Examples of said Angiotensin II receptor antagonists include but are not limited to: (a) Eprosartan mesylate, (b) Irbesartan, (c) Losartan potassium, (d) Olmesartan medoxomil, (e) Telmisartan, (f) Valsartan, and (g) Candesartan Cilexetil.

Examples of said cardioselective beta blockers Include, but are not limited to: (a) Metoprolol succinate and (b) Metoprolol tartrate.

Examples of said lipid regulating drugs (i.e., statins) include, but are not limited to: (a) Atorvastatin calcium, (b) Fluvastatin sodium, (c) Lovastatin, (d) Rosuvastatin calcium, (e) Simvastatin, and (f) Ezetimibe in combination with Simvastatin.

Examples of ACE inhibitors and dosages include, for example:
(a) Benazepril HCl (e.g., Novartis' Lotension brand of Benazepril HCl) administered in amounts of 5 to 40 mg per day,
(b) Captopril tablets (Mylan) administered in amounts of 25 to 300 mg per day,
(c) Moexipril hydrochloride (e.g., Schwartz's Univasc brand of Moexipril hydrochloride) administered in amounts of 7.5 to 30 mg daily,
(d) Perindopril erbumine (e.g., Solvay's Aceron brand of Perindopril erbumine) administered in amounts of 2 to 8 mg a day,
(e) Lisinopril (e.g., Merck's Prinivil brand of Lisinopril) administered in amounts of 10 to 40 mg per day,
(f) Ramipril (e.g., King's Altace brand of Ramipril) administered in amounts of 2.5 to 20 mg once daily,
(g) Trandolapril (e.g., Abbott's Mavik brand of Trandolapril) administered in amounts of 1 to 4 mg daily.

Examples of Angiotensin II receptor antagonists (Angiotensin II receptor blockers) and dosages include, for example:
(a) Eprosartan mesylate (e.g., Kos's Teveten brand of Eprosartan mesylate) administered for a total amount of 400 to 800 mg a day,
(b) Irbesartan (e.g., Sanofi-Aventis' and BMS' Avapro brand of Irbesartan) administered in amounts of 75 to 300 mg daily,
(c) Losartan potassium (e.g., Merck's Cozaar brand of Losartan potassium) administered in a total amount of about 25 to 100 mg daily,
(d) Olmesartan medoxomil (e.g., Daiichi Sankyo's Benicar brand of Olmesartan medoxomil) administered in amounts of 20 to 40 mg once daily,
(e) Telmisartan (e.g., Boehringer Ingelheim's Micardis brand of Telmisartan) administered in amounts of 20 to 80 mg once daily,
(f) Valsartan (e.g., Novartis' Diovan brand of Valsartan) administered in amounts of 80 to 320 mg once per day, and
(g) Candesartan cilexetil (e.g., AstraZeneca's Atacand brand of Candesartan cilexetil) administered in amounts of 2 to 32 mg daily.

Examples of cardioselective Beta blockers and dosages include, for example:
(a) Metoprolol succinate (e.g., Asta Zeneca LP's Toprol-XL brand of Metoprolol succinate) administered in amounts of 25 to 100 mg daily, and
(b) Metoprolol tartrate (e.g., Asta Zeneca LP's Lopressor brand (injection or tablets) of Metoprolol tartrate) administered in amounts of 100 to 450 mg daily.

Examples of said lipid regulating drugs (i.e., statins) and dosages include, for example:
(a) Atorvastatin calcium (e.g., Parke-Davis' Lipitor brand of Atorvastatin calcium) administered in amounts of 10 to 80 mg once daily,
(b) Fluvastatin sodium (e.g., Novartis' Lescol brand of Fluvastatin sodium) administered in amounts of 20 to 80 mg per day,
(c) Lovastatin (e.g., Merck's Mevacor brand of Lovastatin, and Sciele's Altoprev brand of Lovastatin) in amounts of 10 to 80 mg per day,
(d) Rosuvastatin calcium (e.g., AstraZeneca's Crestar brand of Rosuvastatin calcium) administered in amounts of 5 to 40 mg once daily,
(e) Simvastatin (e.g., Merck's Zocor brand of Simvastatin) administered in amounts of 5 to 40 mg a day, and
(f) Ezetimibe in combination with Simvastatin (e.g., Merck's/Schering-Plough Pharmaceuticals' Vytorin brand of Ezetimibe in combination with Simvastatin) administered in amounts of 10 mg per day of Ezetimibe, and 10 to 80 mg per day of Simvastatin.

Determination of the amount of CXCR2 antagonist administered and the amount administered of other drugs selected from the group consisting of: angiotensin-converting enzyme (ACE) inhibitors, Angiotensin II receptor antagonists (Angiotensin II receptor blockers), cardioselective beta blockers, and lipid regulating drugs (statins) is within the judgment of the skilled clinician. Thus, the skilled clinician can adjust the dosages based on the condition of the patient and the patient's responsiveness to the medications administered. The skilled clinician will administer the CXCR2 antagonist, and the other drugs described above in amounts that are sufficient to achieve the desired blood levels of the medications. The amounts of the medications administered are sufficient to reduce or alleviate the symptoms of the chronic obstructive disease and the symptoms of cardiovascular cormobidities present in COPD patients. Thus, the skilled clinician would use a combination of the CXCR2 antagonist and other drugs (described above) in amounts sufficient to treat, alleviate, or reduce symptoms of chronic obstructive pulmonary disease and reduce symptoms and risk of cardiovascular comorbidities in patients with chronic obstructive pulmonary disease, such as ischemic heart disease, systemic arterial hypertension, and peripheral vascular disease.

Thus, one embodiment of this invention is directed to a method of treating chronic obstructive disease in a patient in need of such treatment, said method comprising administering to the patient an effective amount of the CXCR2 antagonist of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg to about 30 mg daily of the CXCR2 antagonist of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg daily of the CXCR2 antagonist of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 10 mg daily of the CXCR2 antagonist of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 30 mg daily of the CXCR2 antagonist of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient an effective amount of the CXCR2 antagonist of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg to about 30 mg daily of the CXCR2 antagonist of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg daily of the CXCR2 antagonist of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 10 mg daily of the CXCR2 antagonist of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 30 mg daily of the CXCR2 antagonist of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient an effective amount of the CXCR2 antagonist of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily of the CXCR2 antagonist of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg daily of the CXCR2 antagonist of formula (1.0A), and administering an effective amount of the lipid regulating drug Simvastatin.

Another embodiment of this invention is directed to a method of treating chronic obstructive disease in a patient in need of such treatment, said method comprising administering to the patient about 10 mg daily of the CXCR2 antagonist of formula (1.0A), and administering an effective amount of the lipid regulating drug Simvastatin.

Another embodiment of this invention is directed to a method of treating chronic obstructive disease in a patient in need of such treatment, said method comprising administering to the patient about 30 mg daily of the CXCR2 antagonist of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily (and in one example 3 mg daily, and in another example 10 mg daily, and in another example 30 mg daily) of the CXCR2 antagonist of formula (1.0A), and administering an effective amount of at least one (e.g., 1, 2 or 3, or 1 or 2, or 1, and usually 1) drug selected from the group consisting of:

(A) angiotensin-converting enzyme (ACE) inhibitors selected from the group consisting of:
(a) Benazepril HO (e.g., Novartis' Lotension brand of Benazepril HCl) administered in amounts of 5 to 40 mg per day,
(b) Captopril tablets (Mylan) administered in amounts of 25 to 300 mg per day,
(c) Moexipril hydrochloride (e.g., Schwartz's Univasc brand of Moexipril hydrochloride) administered in amounts of 7.5 to 30 mg daily,
(d) Perindopril erbumine (e.g., Solvay's Aceron brand of Perindopril erbumine) administered in amounts of 2 to 8 mg a day,
(e) Lisinopril (e.g., Merck's Prinivil brand of Lisinopril) administered in amounts of 10 to 40 mg per day,
(f) Ramipril (e.g., King's Altace brand of Ramipril) administered in amounts of 2.5 to 20 mg once daily,
(g) Trandolapril (e.g., Abbott's Mavik brand of Trandolapril) administered in amounts of 1 to 4 mg daily;

(B) Angiotensin II receptor antagonists (Angiotensin II receptor blockers) selected from the group consisting of:
(a) Eprosartan mesylate (e.g., Kos's Teveten brand of Eprosartan mesylate) administered for a total amount of 400 to 800 mg a day,
(b) Irbesartan (e.g., Sanofi-Aventis' and BMS' Avapro brand of Irbesartan) administered in amounts of 75 to 300 mg daily,
(c) Losartan potassium (e.g., Merck's Cozaar brand of Losartan potassium) administered in a total amount of about 25 to 100 mg daily,
(d) Olmesartan medoxomil (e.g., Daiichi Sankyo's Benicar brand of Olmesartan medoxomil) administered in amounts of 20 to 40 mg once daily,
(e) Telmisartan (e.g., Boehringer Ingelheim's Micardis brand of Telmisartan) administered in amounts of 20 to 80 mg once daily,
(f) Valsartan (e.g., Novartis' Diovan brand of Valsartan) administered in amounts of 80 to 320 mg once per day, and
(g) Candesartan cilexetil (e.g., AstraZeneca's Atacand brand of Candesartan cilexetil) administered in amounts of 2 to 32 mg daily;

(C) Selective Beta blockers selected from the group consisting of:
(a) Metoprolol succinate (e.g., Asta Zeneca LP's Toprol-XL brand of Metoprolol succinate) administered in amounts of 25 to 100 mg daily, and
(b) Metoprolol tartrate (e.g., Asta Zeneca LP's Lopressor brand (injection or tablets) of Metoprolol tartrate) administered in amounts of 100 to 450 mg daily; and

(D) lipid regulating drugs (i.e., statins) selected from the group consisting of:
(a) Atorvastatin calcium (e.g., Parke-Davis' Lipitor brand of Atorvastatin calcium) administered in amounts of 10 to 80 mg once daily,
(b) Fluvastatin sodium (e.g., Novartis' Lescol brand of Fluvastatin sodium) administered in amounts of 20 to 80 mg per day,
(c) Lovastatin (e.g., Merck's Mevacor brand of Lovastatin, and Sciele's Altoprev brand of Lovastatin) in amounts of 10 to 80 mg per day,
(d) Rosuvastatin calcium (e.g., AstraZeneca's Crestor brand of Rosuvastatin calcium) administered in amounts of 5 to 40 mg once daily,
(e) Simvastatin (e.g., Merck's Zocor brand of Simvastatin) administered in amounts of 5 to 40 mg a day, and
(f) Ezetimibe in combination with Simvastatin (e.g., Merck's/Schering-Plough Pharmaceuticals' Vytorin brand of Ezetimibe in combination with Simvastatin) administered in amounts of 10 mg per day of Ezetimibe, and 10 to 80 mg per day of Simvastatin.

Another embodiment of this invention is directed to a method of treating chronic obstructive disease in a patient in need of such treatment, said method comprising administering to the patient an effective amount of the CXCR2 antagonist of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg to about 30 mg daily of the CXCR2 antagonist of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg daily of the CXCR2 antagonist of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 10 mg daily of the CXCR2 antagonist of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 30 mg daily of the CXCR2 antagonist of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient an effective amount of the CXCR2 antagonist of formula (1.0B), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin lI receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg to about 30 mg daily of the CXCR2 antagonist of formula (1.0B), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg daily of the CXCR2 antagonist of formula (1.0B), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 10 mg daily of the CXCR2 antagonist of formula (1.0B), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 30 mg daily of the CXCR2 antagonist of formula (1.0B), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient an effective amount of the CXCR2 antagonist of formula (1.0B), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg to about 30 mg daily of the CXCR2 antagonist of formula (1.0B), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg of the CXCR2 antagonist of formula (1.0B), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 10 mg daily of the CXCR2 antagonist of formula (1.0B), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 30 mg daily of the CXCR2 antagonist of formula (1.0B), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient an effective amount of the CXCR2 antagonist of formula (1.0B), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily of the CXCR2 antagonist of formula (1.0B), and administering an effective amount of the lipid regulating drug Simvastatin.

Another embodiment of this invention is directed to a method of treating chronic obstructive disease in a patient in need of such treatment, said method comprising administering to the patient about 3 mg daily of the CXCR2 antagonist of formula (1.0B), and administering an effective amount of the lipid regulating drug Simvastatin.

Another embodiment of this invention is directed to a method of treating chronic obstructive disease in a patient in need of such treatment, said method comprising administering to the patient about 10 mg daily of the CXCR2 antagonist of formula (1.0B), and administering an effective amount of the lipid regulating drug Simvastatin.

Another embodiment of this invention is directed to a method of treating chronic obstructive disease in a patient in need of such treatment, said method comprising administering to the patient about 30 mg daily of the CXCR2 antagonist of formula (1.0B), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily (and in one example 3 mg daily, and in another example 10 mg daily, and in another example 30 mg daily) of the CXCR2 antagonist of formula (1.0B), and administering an effective amount of at least one (e.g., 1, 2 or 3, or 1 or 2, or 1, and usually 1) drug selected from the group consisting of:

(A) angiotensin-converting enzyme (ACE) inhibitors selected from the group consisting of:
(a) Benazepril HCl (e.g., Novartis' Lotension brand of Benazepril HCl) administered in amounts of 5 to 40 mg per day,
(b) Captopril tablets (Mylan) administered in amounts of 25 to 300 mg per day,
(c) Moexipril hydrochloride (e.g., Schwartz's Univasc brand of Moexipril hydrochloride) administered in amounts of 7.5 to 30 mg daily,
(d) Perindopril erbumine (e.g., Solvay's Aceron brand of Perindopril erbumine) administered in amounts of 2 to 8 mg a day,
(e) Lisinopril (e.g., Merck's Prinivil brand of Lisinopril) administered in amounts of 10 to 40 mg per day,
(f) Ramipril (e.g., King's Altace brand of Ramipril) administered in amounts of 2.5 to 20 mg once daily,
(g) Trandolapril (e.g., Abbott's Mavik brand of Trandolapril) administered in amounts of 1 to 4 mg daily;

(B) Angiotensin II receptor antagonists (Angiotensin II receptor blockers) selected from the group consisting of:
(a) Eprosartan mesylate (e.g., Kos's Teveten brand of Eprosartan mesylate) administered for a total amount of 400 to 800 mg a day,
(b) Irbesartan (e.g., Sanofi-Aventis' and BMS' Avapro brand of Irbesartan) administered in amounts of 75 to 300 mg daily,
(c) Losartan potassium (e.g., Merck's Cozaar brand of Losartan potassium) administered in a total amount of about 25 to 100 mg daily,
(d) Olmesartan medoxomil (e.g., Daiichi Sankyo's Benicar brand of Olmesartan medoxomil) administered in amounts of 20 to 40 mg once daily,
(e) Telmisartan (e.g., Boehringer Ingelheim's Micardis brand of Telmisartan) administered in amounts of 20 to 80 mg once daily,
(f) Valsartan (e.g., Novartis' Diovan brand of Valsartan) administered in amounts of 80 to 320 mg once per day, and
(g) Candesartan cilexetil (e.g. AstraZeneca's Atacand brand of Candesartan cilexetil) administered in amounts of 2 to 32 mg daily;

(C) Cardioselective Beta blockers selected from the group consisting of:
(a) Metoprolol succinate (e.g., Asta Zeneca LP's Toprol-XL brand of Metoprolol succinate) administered in amounts of 25 to 100 mg daily, and
(b) Metoprolol tartrate (e.g., Asta Zeneca LP's Lopressor brand (injection or tablets) of Metoprolol tartrate) administered in amounts of 100 to 450 mg daily; and

(D) lipid regulating drugs (i.e., statins) selected from the group consisting of:
(a) Atorvastatin calcium (e.g., Parke-Davis' Lipitor brand of Atorvastatin calcium) administered in amounts of 10 to 80 mg once daily,
(b) Fluvastatin sodium (e.g., Novartis' Lescol brand of Fluvastatin sodium) administered in amounts of 20 to 80 mg per day,
(c) Lovastatin (e.g., Merck's Mevacor brand of Lovastatin, and Sciefe's Altoprev brand of Lovastatin) in amounts of 10 to 80 mg per day,
(d) Rosuvastatin calcium (e.g., AstraZeneca's Crestor brand of Rosuvastatin calcium) administered in amounts of 5 to 40 mg once daily,
(e) Simvastatin (e.g., Merck's Zocor brand of Simvastatin) administered in amounts of 5 to 40 mg a day, and
(f) Ezetimibe in combination with Simvastatin (e.g., Merck's/Schering-Plough Pharmaceuticals' Vytorin brand of Ezetimibe in combination with Simvastatin) administered in amounts of 10 mg per day of Ezetimibe, and 10 to 80 mg per day of Simvastatin.

Another embodiment of this invention is directed to a method of treating chronic obstructive disease in a patient in need of such treatment, said method comprising administering to the patient an effective amount of the CXCR2 antagonist of formula (1.0B), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg to about 30 mg daily of the CXCR2 antagonist of formula (1.0B), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg daily of the CXCR2 antagonist of formula (1.0B), and administering 5 to 40 mg a day of the lipid regulating drug Simvastatin.

Another embodiment of this invention is directed to a method of treating chronic obstructive disease in a patient in need of such treatment, said method comprising administering to the patient about 10 mg daily of the CXCR2 antagonist of formula (1.0B), and administering 5 to 40 mg a day of the lipid regulating drug Simvastatin.

Another embodiment of this invention is directed to a method of treating chronic obstructive disease in a patient in need of such treatment, said method comprising administering to the patient about 30 mg daily of the CXCR2 antagonist of formula (1.0B), and administering 5 to 40 mg a day of the lipid regulating drug Simvastatin.

Another embodiment of this invention is directed to a method of treating chronic obstructive disease in a patient in need of such treatment, said method comprising administering to the patient an effective amount of a monohydrate of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins),

Another embodiment of this invention is directed to a method of treating chronic obstructive disease in a patient in need of such treatment, said method comprising administering to the patient about 3 mg to about 30 mg daily of a monohydrate of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg daily of a monohydrate of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 10 mg daily of a monohydrate of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 30 mg daily of a monohydrate of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient an effective amount of a monohydrate of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg to about 30 mg daily of a monohydrate of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg daily of a monohydrate of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 10 mg daily of a monohydrate of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 30 mg daily of a monohydrate of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient an effective amount of a monohydrate of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily of a monohydrate of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg daily of a monohydrate of formula (1.0A), and administering an effective amount of the lipid regulating drug Simvastatin.

Another embodiment of this invention is directed to a method of treating chronic obstructive disease in a patient in need of such treatment, said method comprising administering to the patient about 10 mg daily of a monohydrate of formula (1.0A), and administering an effective amount of the lipid regulating drug Simvastatin.

Another embodiment of this invention is directed to a method of treating chronic obstructive disease in a patient in need of such treatment, said method comprising administering to the patient about 30 mg daily of a monohydrate of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily (and in one example 3 mg daily, and in another example 10 mg daily, and in another example 30 mg daily) of a monohydrate of formula (1.0A), and administering an effective amount of at least one (e.g., 1, 2 or 3, or 1 or 2, or 1, and usually 1) drug selected from the group consisting of:

(A) angiotensin-converting enzyme (ACE) inhibitors selected from the group consisting of:
(a) Benazepril HCl (e.g., Novartis' Lotension brand of Benazepril HCl) administered in amounts of 5 to 40 mg per day,
(b) Captopril tablets (Mylan) administered in amounts of 25 to 300 mg per day,
(c) Moexipril hydrochloride (e.g., Schwartz's Univasc brand of Moexipril hydrochloride) administered in amounts of 7.5 to 30 mg daily,
(d) Perindopril erbumine (e.g., Solvay's Aceron brand of Perindopril erbumine) administered in amounts of 2 to 8 mg a day,
(e) Lisinopril (e.g., Merck's Prinivil brand of Lisinopril) administered in amounts of 10 to 40 mg per day,
(f) Ramipril (e.g., King's Altace brand of Ramipril) administered in amounts of 2.5 to 20 mg once daily,
(g) Trandolapril (e.g., Abbott's Mavik brand of Trandolapril) administered in amounts of 1 to 4 mg daily;

(B) Angiotensin II receptor antagonists (Angiotensin II receptor blockers) selected from the group consisting of:
(a) Eprosartan mesylate (e.g., Kos's Teveten brand of Eprosartan mesylate) administered for a total amount of 400 to 800 mg a day,
(b) Irbesartan (e.g., Sanofi-Aventis' and BMS' Avapro brand of Irbesartan) administered in amounts of 75 to 300 mg daily,
(c) Losartan potassium (e.g., Merck's Cozaar brand of Losartan potassium) administered in a total amount of about 25 to 100 mg daily,
(d) Olmesartan medoxomil (e.g., Daiichi Sankyo's Benicar brand of Olmesartan medoxomil) administered in amounts of 20 to 40 mg once daily,
(e) Telmisartan (e.g., Boehringer Ingelheim's Micardis brand of Telmisartan) administered in amounts of 20 to 80 mg once daily,
(f) Valsartan (e.g., Novartis' Diovan brand of Valsartan) administered in amounts of 80 to 320 mg once per day, and
(g) Candesartan cilexetil (e.g., AstraZeneca's Atacand brand of Candesartan cilexetil) administered in amounts of 2 to 32 mg daily;

(C) Cardioselective Beta blockers selected from the group consisting of:
(a) Metoprolol succinate (e.g., Asta Zeneca LP's Toprol-XL brand of Metoprolol succinate) administered in amounts of 25 to 100 mg daily, and
(b) Metoprolol tartrate (e.g., Asta Zeneca LP's Lopressor brand (injection or tablets) of Metoprolol tartrate) administered in amounts of 100 to 450 mg daily; and

(D) lipid regulating drugs (i.e., statins) selected from the group consisting of:
(a) Atorvastatin calcium (e.g., Parke-Davis' Lipitor brand of Atorvastatin calcium) administered in amounts of 10 to 80 mg once daily,
(b) Fluvastatin sodium (e.g., Novartis' Lescol brand of Fluvastatin sodium) administered in amounts of 20 to 80 mg per day,
(c) Lovastatin (e.g., Merck's Mevacor brand of Lovastatin, and Sciele's Altoprev brand of Lovastatin) in amounts of 10 to 80 mg per day,
(d) Rosuvastatin calcium (e.g., AstraZeneca's Creator brand of Rosuvastatin calcium) administered in amounts of 5 to 40 mg once daily,
(e) Simvastatin (e.g., Merck's Zocor brand of Simvastatin) administered in amounts of 5 to 40 mg a day, and
(f) Ezetimibe in combination with Simvastatin (e.g., Merck's/Schering-Plough Pharmaceuticals' Vytorin brand of Ezetimibe in combination with Simvastatin) administered in amounts of 10 mg per day of Ezetimibe, and 10 to 80 mg per day of Simvastatin.

Another embodiment of this invention is directed to a method of treating chronic obstructive disease in a patient in need of such treatment, said method comprising administering to the patient an effective amount of a monohydrate of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg to about 30 mg daily of a monohydrate of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg daily of a monohydrate of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 10 mg daily of a monohydrate of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 30 mg daily of a monohydrate of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient an effective amount of polymorph Form III of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg to about 30 daily mg of polymorph Form III of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg daily of polymorph Form III of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 10 mg daily of polymorph Form III of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin receptor blockers), cardioselective beta blockers, and lipid regulating drugs (statins).

Another embodiment of this invention is directed to a method of treating chronic obstructive disease in a patient in need of such treatment, said method comprising administering to the patient about 30 mg daily of polymorph Form III of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient an effective amount of polymorph Form III of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg to about 30 mg daily of polymorph Form III of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg daily of polymorph Form III of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 10 mg daily of polymorph Form III of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 30 mg daily of polymorph Form III of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient an effective amount of polymorph Form III of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily of polymorph Form III of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg daily of polymorph Form III of formula (1.0A), and administering an effective amount of the lipid regulating drug Simvastatin.

Another embodiment of this invention is directed to a method of treating chronic obstructive disease in a patient in need of such treatment, said method comprising administering to the patient about 10 mg daily of polymorph Form III of formula (1.0A), and administering an effective amount of the lipid regulating drug Simvastatin.

Another embodiment of this invention is directed to a method of treating chronic obstructive disease in a patient in need of such treatment, said method comprising administering to the patient about 30 mg daily of polymorph Form III of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily (and in one example 3 mg daily, and in another example 10 mg daily, and in another example 30 mg daily) of polymorph Form III of formula (1.0A), and administering an effective amount of at least one (e.g., 1, 2 or 3, or 1 or 2, or 1, and usually 1) drug selected from the group consisting of:

(A) angiotensin-converting enzyme (ACE) inhibitors selected from the group consisting of:
(a) Benazepril HCl (e.g., Novartis' Lotension brand of Benazepril HCl) administered in amounts of 5 to 40 mg per day,
(b) Captopril tablets (Mylan) administered in amounts of 25 to 300 mg per day,
(c) Moexipril hydrochloride (e.g., Schwartz's Univasc brand of Moexipril hydrochloride) administered in amounts of 7.5 to 30 mg daily,
(d) Perindopril erbumine (e.g., Solvay's Aceron brand of Perindopril erbumine) administered in amounts of 2 to 8 mg a day,
(e) Lisinopril (e.g., Merck's Prinivil brand of Lisinopril) administered in amounts of 10 to 40 mg per day,
(f) Ramipril (e.g., King's Altace brand of Ramipril) administered in amounts of 2.5 to 20 mg once daily,
(g) Trandolapril (e.g., Abbott's Mavik brand of Trandolapril) administered in amounts of 1 to 4 mg daily;

(B) Angiotensin II receptor antagonists (Angiotensin II receptor blockers) selected from the group consisting of:
(a) Eprosartan mesylate (e.g., Kos's Teveten brand of Eprosartan mesylate) administered for a total amount of 400 to 800 mg a day,
(b) Irbesartan (e.g., Sanofi-Aventis' and BMS' Avapro brand of Irbesartan) administered in amounts of 75 to 300 mg daily,
(c) Losartan potassium (e.g., Merck's Cozaar brand of Losartan potassium) administered in a total amount of about 25 to 100 mg daily,
(d) Olmesartan medoxomil (e.g., Daiichi Sankyo's Benicar brand of Olmesartan medoxomil) administered in amounts of 20 to 40 mg once daily,
(e) Telmisartan (e.g., Boehringer Ingelheim's Micardis brand of Telmisartan) administered in amounts of 20 to 80 mg once daily,
(f) Valsartan (e.g., Novartis' Diovan brand of Valsartan) administered in amounts of 80 to 320 mg once per day, and
(g) Candesartan cilexetil (e.g., AstraZeneca's Atacand brand of Candesartan cilexetil) administered in amounts of 2 to 32 mg daily;

(C) Cardioselective Beta blockers selected from the group consisting of:
(a) Metoprolol succinate (e.g., Asta Zeneca LP's Toprol-XL brand of Metoprolol succinate) administered in amounts of 25 to 100 mg daily, and
(b) Metoprolol tartrate (e.g., Asta Zeneca LP's Lopressor brand (injection or tablets) of Metoprolol tartrate) administered in amounts of 100 to 450 mg daily; and

(D) lipid regulating drugs (i.e., statins) selected from the group consisting of:
(a) Atorvastatin calcium (e.g., Parke-Davis' Lipitor brand of Atorvastatin calcium) administered in amounts of 10 to 80 mg once daily,
(b) Fluvastatin sodium (e.g., Novartis' Lescol brand of Fluvastatin sodium) administered in amounts of 20 to 80 mg per day,
(c) Lovastatin (e.g., Merck's Mevacor brand of Lovastatin, and Sciele's Altoprev brand of Lovastatin) in amounts of 10 to 80 mg per day,
(d) Rosuvastatin calcium (e.g., AstraZeneca's Crestor brand of Rosuvastatin calcium) administered in amounts of 5 to 40 mg once daily,
(e) Simvastatin (e.g., Merck's Zocor brand of Simvastatin) administered in amounts of 5 to 40 mg a day, and
(f) Ezetimibe in combination with Simvastatin (e.g., Merck's/Schering-Plough Pharmaceuticals' Vytorin brand of Ezetimibe in combination with Simvastatin) administered in amounts of 10 mg per day of Ezetimibe, and 10 to 80 mg per day of Simvastatin.

Another embodiment of this invention is directed to a method of treating chronic obstructive disease in a patient in need of such treatment, said method comprising administering to the patient an effective amount of polymorph Form III of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg to about 30 mg daily of polymorph Form III of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg daily of polymorph Form III of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 10 mg daily of polymorph Form III of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 30 mg daily of polymorph Form III of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient an effective amount of polymorph Form IV of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg to about 30 mg daily of polymorph Form IV of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 3 mg daily of polymorph Form IV of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin receptor blockers), cardioselective beta blockers, and lipid regulating drugs (statins).

Another embodiment of this invention is directed to a method of treating chronic obstructive disease in a patient in need of such treatment, said method comprising administering to the patient about 10 mg daily of polymorph Form IV of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient about 30 mg daily of polymorph Form IV of formula (1.0A), and administering an effective amount of at least one (e.g., 1, 2 or 3, or 1 or 2, or 1, and usually 1) drug selected from the group consisting of: angiotensin-converting enzyme (ACE) inhibitors, Angiotensin II receptor antagonists (Angiotensin II receptor blockers), cardioselective beta blockers, and lipid regulating drugs (statins).

Another embodiment of this invention is directed to a method of treating chronic obstructive disease in a patient in need of such treatment, said method comprising administering to the patient an effective amount of polymorph Form IV of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg to about 30 mg daily of polymorph Form IV of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 3 mg daily of polymorph Form IV of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 10 mg daily of polymorph Form IV of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient about 30 mg daily of polymorph Form IV of formula (1.0A), and administering an effective amount of at least one (e.g., 1, 2 or 3, or 1 or 2, or 1, and usually 1) lipid regulating drugs (statins).

Another embodiment of this invention is directed to a method of treating chronic obstructive disease in a patient in need of such treatment, said method comprising administering to the patient an effective amount of polymorph Form IV of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily of polymorph Form IV of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg daily of polymorph Form IV of formula (1.0A), and administering an effective amount of the lipid regulating drug Simvastatin.

Another embodiment of this invention is directed to a method of treating chronic obstructive disease in a patient in need of such treatment, said method comprising administering to the patient about 10 mg daily of polymorph Form IV of formula (1.0A), and administering an effective amount of the lipid regulating drug Simvastatin.

Another embodiment of this invention is directed to a method of treating chronic obstructive disease in a patient in need of such treatment, said method comprising administering to the patient about 30 mg daily of polymorph Form IV of formula (1.0A), and administering an effective amount of the lipid regulating drug Simvastatin.

Another embodiment of this invention is directed to a method of treating chronic obstructive disease in a patient in need of such treatment, said method comprising administering to the patient about 3 mg to about 30 mg daily (and in one example 3 mg daily, and in another example 10 mg daily, and in another example 30 mg daily) of polymorph Form IV of formula (1.0A), and administering an effective amount of at least one (e.g., 1, 2 or 3, or 1 or 2, or 1, and usually 1) drug selected from the group consisting of:

(A) angiotensin-converting enzyme (ACE) inhibitors selected from the group consisting of:
(a) Benazepril HCl (e.g., Novartis' Lotension brand of Benazepril HCl) administered in amounts of 5 to 40 mg per day,
(b) Captopril tablets (Mylan) administered in amounts of 25 to 300 mg per day,
(c) Moexipril hydrochloride (e.g., Schwartz's Univasc brand of Moexipril hydrochloride) administered in amounts of 7.5 to 30 mg daily,
(d) Perindopril erbumine (e.g., Solvay's Aceron brand of Perindopril erbumine) administered in amounts of 2 to 8 mg a day,
(e) Lisinopril (e.g., Merck's Prinivil brand of Lisinopril) administered in amounts of 10 to 40 mg per day,
(f) Ramipril (e.g., King's Altace brand of Ramipril) administered in amounts of 2.5 to 20 mg once daily,
(g) Trandolapril (e.g., Abbott's Mavik brand of Trandolapril) administered in amounts of 1 to 4 mg daily;

(B) Angiotensin II receptor antagonists (Angiotensin II receptor blockers) selected from the group consisting of:
(a) Eprosartan mesylate (e.g., Kos's Teveten brand of Eprosartan mesylate) administered for a total amount of 400 to 800 mg a day,
(b) Irbesartan (e.g., Sanofi-Aventis' and BMS' Avapro brand of Irbesartan) administered in amounts of 75 to 300 mg daily,
(c) Losartan potassium (e.g., Merck's Cozaar brand of Losartan potassium) administered in a total amount of about 25 to 100 mg daily,
(d) Olmesartan medoxomil (e.g., Daiichi Sankyo's Benicar brand of Olmesartan medoxomil) administered in amounts of 20 to 40 mg once daily,
(e) Telmisartan (e.g., Boehringer Ingelheim's Micardis brand of Telmisartan) administered in amounts of 20 to 80 mg once daily,
(f) Valsartan (e.g., Novartis' Diovan brand of Valsartan) administered in amounts of 80 to 320 mg once per day, and
(g) Candesartan cilexetil (e.g., AstraZeneca's Atacand brand of Candesartan cilexetil) administered in amounts of 2 to 32 mg daily;

(C) Cardioselective Beta blockers selected from the group consisting of:
(c) Metoprolol succinate (e.g., Asta Zeneca LP's Toprol-XL brand of Metoprolol succinate) administered in amounts of 25 to 100 mg daily, and
(d) Metoprolol tartrate (e.g., Asta Zeneca LP's Lopressor brand (injection or tablets) of Metoprolol tartrate) administered in amounts of 100 to 450 mg daily; and

(D) lipid regulating drugs (i.e., statins) selected from the group consisting of:
(a) Atorvastatin calcium (e.g., Parke-Davis' Lipitor brand of Atorvastatin calcium) administered in amounts of 10 to 80 mg once daily,
(b) Fluvastatin sodium (e.g., Novartis' Lescol brand of Fluvastatin sodium) administered in amounts of 20 to 80 mg per day,
(c) Lovastatin (e.g., Merck's Mevacor brand of Lovastatin, and Sciele's Altoprev brand of Lovastatin) in amounts of 10 to 80 mg per day,
(d) Rosuvastatin calcium (e.g., AstraZeneca's Crestar brand of Rosuvastatin calcium) administered in amounts of 5 to 40 mg once daily,
(e) Simvastatin (e.g., Merck's Zocor brand of Simvastatin) administered in amounts of 5 to 40 mg a day, and
(f) Ezetimibe in combination with Simvastatin (e.g., Merck's/Schering-Plough Pharmaceuticals' Vytorin brand of Ezetimibe in combination with Simvastatin) administered in amounts of 10 mg per day of Ezetimibe, and 10 to 80 mg per day of Simvastatin.

Another embodiment of this invention is directed to a method of treating chronic obstructive disease in a patient in need of such treatment, said method comprising administering to the patient an effective amount of polymorph Form IV of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg to about 30 mg daily of polymorph Form IV of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 3 mg daily of polymorph Form IV of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 10 mg daily of polymorph Form IV of formula (1.0A), and administering 5 to 40 mg a day of the lipid regulating drug Simvastatin.

Another embodiment of this invention is directed to a method of treating chronic obstructive disease in a patient in need of such treatment, said method comprising administering to the patient about 30 mg daily of polymorph Form IV of formula (1.0A), and administering 5 to 40 mg a day of the lipid regulating drug Simvastatin.

Other embodiments of this invention are directed to any one of the above method embodiments, wherein said compound of formula (1.0A) (or monohydrate, or polymorph of Form III or IV) is administered as a pharmaceutical composition, said composition comprising said compound of formula (1.0A) and a pharmaceutically acceptable carrier.

Other embodiments of this invention are directed to any one of the above method embodiments wherein said compound of formula (1.0B) is administered as a pharmaceutical composition, said composition comprising said compound of formula (1.0B), and a pharmaceutically acceptable carrier.

Other embodiments of this invention are directed to any one of the above method embodiments using a compound of formula (1.0A) wherein said compound of formula (1.0A) and the other drugs used are administered in the same pharmaceutical composition (i.e., the same dosage form).

Other embodiments of this invention are directed to any one of the above method embodiments using a monohydrate of formula (1.0A) wherein said monohydrate and the other drugs used are administered in the same pharmaceutical composition (i.e., the same dosage form).

Other embodiments of this invention are directed to any one of the above method embodiments using a polymorph Form III of formula (1.0A) wherein said polymorph and the other drugs used are administered in the same pharmaceutical composition (i.e., the same dosage form).

Other embodiments of this invention are directed to any one of the above method embodiments using a polymorph Form IV of formula (1.0A) wherein said polymorph and the other drugs used are administered in the same pharmaceutical composition (i.e., the same dosage form).

Another embodiment of this invention is directed to a pharmaceutical composition comprising an effective amount (e.g., 3 mg to 30 mg, and in one example 3 mg, and in another example 10 mg, and in another example 30 mg) of a compound of formula (1.0A), a pharmaceutically acceptable carrier, and at least one (e.g., 1, 2 or 3, or 1 or 2, or 1, and usually 1) drug selected from the group consisting of:

(A) angiotensin-converting enzyme (ACE) inhibitors selected from the group consisting of: (a) Benazepril HCl, (b) Captopril, (c) Moexipril hydrochloride, (d) Perindopril erbumine, (e) Lisinopril, (f) Ramipril, and (g) Trandolapril;

(B) Angiotensin II receptor antagonists (Angiotensin II receptor blockers) selected from the group consisting of: (a) Eprosartan mesylate, (b) Irbesartan, (c) Losartan potassium, (d) Olmesartan medoxomil, (e) Telmisartan, (f) Valsartan, and (g) Candesartan cilexetil;

(C) Cardioselective Beta blockers selected from the group consisting of: (a) Metoprolol succinate, and (b) Metoprolol tartrate; and

(D) lipid regulating drugs (i.e., statins) selected from the group consisting of: (a) Atorvastatin calcium, (b) Fluvastatin sodium, (c) Lovastatin, (d) Rosuvastatin calcium, (e) Simvastatin, and (f) Ezetimibe in combination with Simvastatin.

Another embodiment of this invention is directed to a pharmaceutical composition comprising an effective amount (e.g., 3 mg to 30 mg, and in one example 3 mg, and in another example 10 mg, and in another example 30 mg) of a monohydrate of formula (1.0A), a pharmaceutically acceptable carrier, and at least one (e.g., 1, 2 or 3, or 1 or 2, or 1, and usually 1) drug selected from the group consisting of:

(A) angiotensin-converting enzyme (ACE) inhibitors selected from the group consisting of: (a) Benazepril HCl, (b) Captopril, (c) Moexipril hydrochloride, (d) Perindopril erbumine, (e) Lisinopril, (f) Ramipril, and (g) Trandolapril;

(B) Angiotensin II receptor antagonists (Angiotensin II receptor blockers) selected from the group consisting of: (a) Eprosartan mesylate, (b) Irbesartan, (c) Losartan potassium, (d) Olmesartan medoxomil, (e) Telmisartan, (f) Valsartan, and (g) Candesartan cilexetil;

(C) Cardioselective Beta blockers selected from the group consisting of: (a) Metoprolol succinate, and (b) Metoprolol tartrate; and

(D) lipid regulating drugs (i.e., statins) selected from the group consisting of: (a) Atorvastatin calcium, (b) Fluvastatin sodium, (c) Lovastatin, (d) Rosuvastatin calcium, (e) Simvastatin, and (f) Ezetimibe in combination with Simvastatin.

Another embodiment of this invention is directed to a pharmaceutical composition comprising an effective amount amount (e.g., 3 mg to 30 mg, and in one example 3 mg, and in another example 10 mg, and in another example 30 mg) of polymorph form III of formula (1.0A), a pharmaceutically acceptable carrier, and at least one (e.g., 1, 2 or 3, or 1 or 2, or 1, and usually 1) drug selected from the group consisting of:

(A) angiotensin-converting enzyme (ACE) inhibitors selected from the group consisting of: (a) Benazepril HCl, (b) Captopril, (c) Moexipril hydrochloride, (d) Perindopril erbumine, (e) Lisinopril, (f) Ramipril, and (g) Trandolapril;

(B) Angiotensin II receptor antagonists (Angiotensin II receptor blockers) selected from the group consisting of: (a) Eprosartan mesylate, (b) Irbesartan, (c) Losartan potassium, (d) Olmesartan medoxomil, (e) Telmisartan, (f) Valsartan, and (g) Candesartan cilexetil;

(C) Cardioselective Beta blockers selected from the group consisting of: (a) Metoprolol succinate, and (b) Metoprolol tartrate; and

(D) lipid regulating drugs (i.e., statins) selected from the group consisting of: (a) Atorvastatin calcium, (b) Fluvastatin sodium, (c) Lovastatin, (d) Rosuvastatin calcium, (e) Simvastatin, and (f) Ezetimibe in combination with Simvastatin.

Another embodiment of this invention is directed to a pharmaceutical composition comprising an effective amount (e.g., 3 mg to 30 mg, and in one example 3 mg, and in another example 10 mg, and in another example 30 mg) of polymorph Form IV of formula (1.0A), a pharmaceutically acceptable carrier, and at least one (e.g., 1, 2 or 3, or 1 or 2, or 1, and usually 1) drug selected from the group consisting of:

(A) angiotensin-converting enzyme (ACE) inhibitors selected from the group consisting of: (a) Benazepril HCl, (b) Captopril, (c) Moexipril hydrochloride, (d) Perindopril erbumine, (e) Lisinopril, (f) Ramipril, and (g) Trandolapril;

(B) Angiotensin II receptor antagonists (Angiotensin II receptor blockers) selected from the group consisting of: (a) Eprosartan mesylate, (b) Irbesartan, (c) Losartan potassium, (d) Olmesartan medoxomil, (e) Telmisartan, (f) Valsartan, and (g) Candesartan cilexetil;

(C) Cardioselective Beta blockers selected from the group consisting of: (a) Metoprolol succinate, and (b) Metoprolol tartrate; and

(D) lipid regulating drugs (i.e., statins) selected from the group consisting of: (a) Atorvastatin calcium, (b) Fluvastatin sodium, (c) Lovastatin, (d) Rosuvastatin calcium, (e) Simvastatin, and (f) Ezetimibe in combination with Simvastatin.

Another embodiment of this invention is directed to a pharmaceutical composition comprising an effective amount (e.g., 3 mg to 30 mg, and in one example 3 mg, and in another example 10 mg, and in another example 30 mg) of a compound of formula (1.0A), a pharmaceutically acceptable carrier, and at least one (e.g., 1, 2 or 3, or 1 or 2, or 1, and usually 1) lipid regulating drug (i.e., statin) selected from the group consisting of: (a) Atorvastatin calcium, (b) Fluvastatin sodium, (c) Lovastatin, (d) Rosuvastatin calcium, (e) Simvastatin, and (f) Ezetimibe in combination with Simvastatin

Another embodiment of this invention is directed to a pharmaceutical composition comprising an effective amount (e.g., 3 mg to 30 mg, and in one example 3 mg, and in another example 10 mg, and in another example 30 mg) of a compound of formula (1.0A), a pharmaceutically acceptable carrier and an effective amount of Simvastatin.

Another embodiment of this invention is directed to a pharmaceutical composition comprising an effective amount (e.g., 3 mg to 30 mg, and in one example 3 mg, and in another example 10 mg, and in another example 30 mg) of a monohydrate of formula (1.0A), a pharmaceutically acceptable carrier and an effective amount of Simvastatin.

Another embodiment of this invention is directed to a pharmaceutical composition comprising an effective amount (e.g., 3 mg to 30 mg, and in one example 3 mg, and in another example 10 mg, and in another example 30 mg) of polymorph Form III of formula (1.0A), a pharmaceutically acceptable carrier and an effective amount of Simvastatin.

Another embodiment of this invention is directed to a pharmaceutical composition comprising an effective amount (e.g., 3 mg to 30 mg, and in one example 3 mg, and in another example 10 mg, and in another example 30 mg) of polymorph Form IV of formula (1.0A), a pharmaceutically acceptable carrier and an effective amount of Simvastatin.

Other embodiments of this invention are directed to any one of the above embodiments directed to a pharmaceutical composition comprising: (1) a compound of formula (1.0A) (or monohydrate thereof, or polymorph Form III thereof, or polymorph Form IV thereof), and (2) Simvastatin, wherein said Simvastatin is present in amounts of 5 to 40 mg.

Other embodiments of this invention are directed to any one of the above embodiments directed to a pharmaceutical composition comprising: (1) a compound of formula (1.0A) (or monohydrate thereof, or polymorph Form III thereof, or polymorph Form IV thereof), and (2) at least one drug selected from the group consisting of: angiotensin-converting enzyme (ACE) inhibitors, Angiotensin II receptor antagonists (Angiotensin II receptor blockers), Cardioselective Beta blockers, and lipid regulating drugs (i.e., statins), wherein:

(A) said angiotensin-converting enzyme (ACE) inhibitors are selected from the group consisting of: (a) 5 to 40 mg of Benazepril HCl, (b) 25 to 300 mg of Captopril, (c) 7.5 to 30 mg of Moexipril hydrochloride, (d) 2 to 8 mg of Perindopril erbumine, (e) 10 to 40 mg Lisinopril, (f) 2.5 to 20 mg Ramipril, and (g) 1 to 4 mg Trandolapril;

(B) said Angiotensin II receptor antagonists (Angiotensin II receptor blockers) selected from the group consisting of: (a) 400 to 800 mg Eprosartan mesylate, (b) 75 to 300 mg Irbesartan, (c) 25 to 100 mg Losartan potassium, (d) 20 to 40 mg Olmesartan medoxomil, (e) 20 to 80 mg Telmisartan, (f) 80 to 320 mg Valsartan, and (g) 2 to 32 mg Candesartan cilexetil;

(C) said cardioselective Beta blockers selected from the group consisting of: (a) 25 to 100 mg Metoprolol succinate, and (b) 100 to 450 mg Metoprolol tartrate; and

(D) said lipid regulating drugs (i.e., statins) selected from the group consisting of: (a) 10 to 80 mg Atorvastatin calcium, (b) 20 to 80 mg Fluvastatin sodium, (c) 10 to 80 mg Lovastatin, (d) 5 to 40 mg Rosuvastatin calcium, (e) 5 to 40 mg Simvastatin, and (f) 10 mg Ezetimibe and 10 to 80 mg Simvastatin.

Other embodiments of this invention are directed to any one of the embodiments using polymorph Form III of formula (1.0A), except that polymorph Form I is used instead of polymorph Form III.

Other embodiments of this invention are directed to any one of the embodiments using polymorph Form III of formula (1.0A), except that polymorph Form II is used instead of polymorph Form

Those skilled in the art will appreciate that the amounts specified in the pharmaceutical compositions are the amounts per dosage form.

In one example of the embodiments of this invention, a capsule is the dosage form used.

In another example of the embodiments of this invention, a tablet is the dosage form used.

In the methods of this invention the compound of formula (1.0A) (or a monohydrate thereof, or a polymorph thereof) or the compound of formula (1.0B) are usually administered as a separate pharmaceutical composition (i.e., a separate dosage form), and the angiotensin-converting enzyme (ACE) inhibitors, Angiotensin II receptor antagonists (Angiotensin II receptor blockers), cardioselective Beta blockers, and lipid regulating drugs (i.e., statins) are usually administered in their separate dosage forms. In these embodiments the separate dosage forms can be administered simultaneously (i.e., concurrently), or consecutively.

Separate pharmaceutical compositions comprising the compound of formula (1.0A) are described below.

Unless indicated otherwise, the following definitions apply throughout the present specification and claims. These definitions apply regardless of whether a term is used by itself or in combination with other terms.

“An effective amount” means a therapeutically effective amount. An effective amount is that amount that provides the desired blood levels (e.g., the desired pK) of the active ingredients, such that there is a therapeutic benefit to the patient. For example, “an effective amount” is that amount that alleviates the symptoms of COPD,

“At least one” represents, for example, 1, or 1 or 2, or 1, 2 or 3.

“One or more” represents, for example, 1, 1 or 2, or 1, 2 or 3.

“Patient” includes both human and other mammals, preferably human.

“Mammal” includes a human being, and preferably means a human being.

“Composition” is intended to encompass a product comprising the specified ingredients in the specified amounts, as well as any product which results, directly or indirectly, from combination of the specified ingredients in the specified amounts.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal composition can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated.

In the methods of this invention the compound of formula (1.0A) (or a monohydrate thereof, or a polymorph thereof) or the compound of formula (1.0B) are usually administered as a separate pharmaceutical composition (i.e., a separate dosage form), and the angiotensin-converting enzyme (ACE) inhibitors, Angiotensin II receptor antagonists (Angiotensin II receptor blockers), cardioselective Beta blockers, and lipid regulating drugs (i.e., statins) are usually administered in their separate dosage forms. In these embodiments the separate dosage forms can be administered simultaneously (i.e., concurrently), or consecutively.

Separate pharmaceutical compositions comprising the compound of formula (1.0A) are described below.

The pharmaceutical composition comprising the compound of formula (1.0A) (or a pharmaceutically acceptable salt thereof) also comprises at least one pharmaceutically acceptable excipient.

The pharmaceutical composition comprising the compound of formula (1.0A), or a pharmaceutically acceptable salt thereof, and at least one pharmaceutically acceptable excipient provides release of at least about 83% of the compound of formula (1.0A) in 5 minutes when tested using a USPII Paddle Stirrer apparatus filled with 900 mL of dissolution medium consisting of 0.5% sodium lauryl sulfate solution buffered with pH 6.8 sodium phosphate buffer at 37° C.±0.5° C. with the paddle speed set at 75 RPM. Preferably, the composition provides release of at least about 99% of the compound of formula (1.0A) in 15 minutes.

In one embodiment, at least one pharmaceutically acceptable excipient is one or more wetting agent(s), one or more binder(s), one or more diluent(s), or one or more disintegrant(s). In another embodiment, at least one pharmaceutically acceptable excipient is one or more wetting agent(s), one or more binder(s), one or more diluent(s), and one or more disintegrant(s). In yet another embodiment, at least one pharmaceutically acceptable excipient is a wetting agent, a binder, a diluent, or a disintegrant, or any combination of two or more thereof.

As used herein the phrase “pharmaceutically acceptable salt” refers to a non-toxic salt prepared from a pharmaceutically acceptable acid or base (including inorganic acids or bases, or organic acids or bases). Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine, and procaine.

In one embodiment the pharmaceutically acceptable salts of the compound of formula (1.0A) can be prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

As used herein the term “capsule” refers to a special container or enclosure made of methyl cellulose, polyvinyl alcohols, or denatured gelatins or starch for holding or containing a composition comprising a composition of the present invention and a carrier. There are soft shell gel capsules and hard shell gel capsules. In contrast to soft shell gel capsules, hard shell gel capsules are typically made of blends of relatively high gel strength bone and pork skin gelatins. The capsule itself may contain small amounts of dyes, opaquing agents, plasticizers, and preservatives.

As used herein the term “tablet” refers to an orally disintegrating tablet containing a composition comprising a composition of the present invention and a carrier with suitable diluents. The tablet can be prepared by soft compression of mixtures or granulations or by lyophilization.

As used herein the phrase “oral gel” refers to a composition comprising a composition of the present invention and a carrier dispersed or solubilized in a hydrophilic semi-solid matrix.

As used herein the phrase “orally consumable film” refers to a composition comprising a composition of the present invention and an edible film carrier.

As used herein the phrase “powders for constitution” refers to powder blends containing a composition comprising a composition of the present invention and a carrier with suitable diluents which can be suspended in water or juices.

As used herein the term “diluent” refers to a substance that usually makes up the major portion of the composition. Suitable diluents include sugars such as lactose, sucrose, mannitol, and sorbitol; starches derived from wheat, corn rice, and potato; and celluloses such as microcrystalline cellulose. The amount of diluent in the composition can range from about 10% to about 90% by weight of the total composition, preferably from about 25% to about 90% by weight, more preferably from about 25% to about 80%, more preferably from about 30% to about 80% by weight, even more preferably from about 65% to about 80% by weight.

As used herein the term “disintegrant” refers to a substance added to the composition to help it break apart (disintegrate) and release the medicinal agent(s). Suitable disintegrants include starches; “cold water soluble” modified starches such as sodium carboxymethyl starch; natural and synthetic gums such as locust bean, karaya, guar, tragacanth, and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; effervescent mixtures; and super-disintegrants such as sodium starch glycolate, crospovidone, and croscarmellose sodium. The amount of disintegrant in the composition can range from about 2% to about 30% by weight of the composition, preferably from about 4% to about 22% by weight, more preferably from about 4% to about 17% by weight, even more preferably from about 4% to about 15% by weight.

As used herein the term “binder” refers to a substance that binds or “glues” powders together and makes them cohesive by forming granules, thus serving as the “adhesive” in the composition. Binders add cohesive strength already available in the diluent or bulking agent. Suitable binders include sugars such as sucrose; starches derived from wheat, corn rice, and potato, including pregelatinized starch; natural gums such as acacia, gelatin, and tragacanth; derivatives of seaweed such as alginic acid, sodium alginate, and ammonium calcium alginate; cellulosic materials such as methylcellulose, sodium carboxymethylcellulose, and hydroxypropylmethylcellulose; polyvinylpyrrolidinone; and inorganics such as magnesium aluminum silicate. The amount of binder in the composition can range from about 0.1% to about 20% by weight of the composition, preferably from about 0.3% to about 10% by weight, more preferably 0.3% to about 5% by weight, even more preferably from about 0.3% to about 3% by weight.

As used herein the term “lubricant” refers to a substance added to the composition to enable the granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols, and d'l-leucine. Lubricants are usually added at the very last step before compression, since they must be present on the surfaces of the granules and in between them and the parts of the tablet press. The amount of lubricant in the composition can range from about 0.2% to about 5% by weight of the composition, preferably from about 0.5% to about 2%, more preferably from about 0.3% to about 1.5% by weight.

As used herein the term “glidant” refers to a substance that prevents caking and improves the flow characteristics of granulations, so that flow is smooth and uniform. Suitable glidants include silicon dioxide and talc. The amount of glidant in the composition can range from about 0.1% to about 5% by weight of the total composition, preferably from about 0.5% to about 2% by weight.

As used herein the phrase “wetting agent” refers to a substance that allows the composition to be wetted by lowering its surface tension. Wetting agents may be anionic, cationic, or nonionic. Suitable wetting agents include docusate sodium, emulsifying wax. BP, self-emulsifying glyceryl monooleate, sodium lauryl sulfate, benzethonium chloride, cetrimide, sodium lauryl sulfate incompatibility, chlorhexidine activity, emulsifying waxes, butylparaben, emulsifying wax USP, ethylparaben, glyceryl monooleate, methylparaben, polyoxyethylene alkyl ethers, polyoxyethylene castor oil derivatives, polyoxyethylene sorbitan fatty acid esters, polyoxyethylene stearates, polysorbate 80, propylparaben, sorbic acid, sorbitan esters, and triethyl citrate. The amount of the wetting agent can vary from about 0.1% to about 8% by weight of the composition, more preferably, 0.1% to about 5% by weight of the composition, yet more preferably from about 0.1% to about 1%.

In one embodiment of the composition, one or more wetting agent(s), one or more binder(s), one or more diluent(s), and one or more disintegrant(s) are blended in a fluid bed. In one embodiment, one or more welling agent(s) is poloxamer present in a ratio of poloxamer to the compound of formula (1.0A) of between about 0.3:1 to about 1.2:1. Preferably, the ratio of poloxamer to the compound of formula (1.0A) is about 1.2 to 1. In one embodiment, one or more wetting agent(s) is poloxamer present at about 0.1-8% (w/w).

In one embodiment, one or more binder(s) is present at about 0.1% to about 20% (w/w). In one preferred embodiment, one or more binder(s) is povidone present in a ratio of povidone to the compound of formula (1.0A) of between about 0.18:1 to about 1.8:1. In another preferred embodiment, the ratio of povidone to the compound of formula (1.0A) is about 0.66 to 1. In one embodiment, one or more binder(s) is povidone present at about 0.3% to about 5% (w/w). In one embodiment, one or more binder(s) is povidone present at about 2% to about 3% (w/w).

In one embodiment, the composition is stable for at least 6 months at 40° C./75% relative humidity (RH) when packaged in high density polyethylene bottles (HDPE) bottles. Preferably, the composition is stable for at least 18 months at 25° C./60% RH when packaged in high density polyethylene bottles (HDPE) bottles.

In one embodiment, one or more binder(s) is pregelatinized starch present at about 0.1% to about 20% (w/w). In one preferred embodiment, pregelatinized starch is present at a ratio of pregelatinized starch to the compound of formula (1.0A) of between about 0.3:1 to about 1.2:1. In another preferred embodiment, pregelatinized starch is present at about 6% to about 7% (w/w).

In one embodiment, one or more diluent(s) is present at about 10% to about 90% (w/w). In one preferred embodiment, one or more diluent(s) is microcrystalline cellulose and lactose.

In one embodiment, one or more disintegrant(s) is present at about 2% to about 30% (w/w). In one preferred embodiment, one or more disintegrant(s) is crospovidone.

In one embodiment, the composition further comprises one or more glidant(s), In one preferred embodiment, one or more glidant(s) is present at about 0.1% to about 5% (w/w). In one preferred embodiment, one or more glidant(s) is silicon dioxide.

In one embodiment, the composition further comprises one or more lubricant(s).

Preferably, one or more lubricant(s) is present at about 0.2% to about 5% (w/w). In one preferred embodiment, one or more lubricant(s) is magnesium stearate.

In one embodiment the composition comprises:

| | mg per unit |
|---|---|
| | dose of |
| Components | composition |
| Formula (1.0A) | 3 |
| monohydrate | |
| Lactose Monohydrate | 115.72 |
| Microcrystalline Cellulose | 35.2 |
| Crospovidone | 16.5 |
| Povidone | 5.28 |
| Sodium Lauryl Sulfate | 0.3 |

In another embodiment, the composition further comprises the following components:

| | mg per unit |
|---|---|
| | dose of |
| Components | composition |
| Microcrystalline Cellulose | 24.2 |
| Crospovidone | 16.5 |

In another embodiment, the composition further comprises the following components:

| | mg per unit dose of |
|---|---|
| Components | composition |
| Silicon Dioxide | 2.2 |
| Magnesium | 1.1 |
| Stearate | |

In another embodiment the composition comprises the following components:

| | mg per unit dose of |
|---|---|
| Components | composition |
| Formula (1.0A) | 10 |
| monohydrate | |
| Lactose Monohydrate | 108.02 |
| Microcrystalline Cellulose | 35.2 |
| Crospovidone | 16.5 |
| Povidone | 5.28 |
| Sodium Lauryl Sulfate | 1 |

In one embodiment, the composition further comprises the following components:

| | mg per unit dose of |
|---|---|
| Components | composition |
| Microcrystalline Cellulose | 24.2 |
| Crospovidone | 16.5 |

In one embodiment, the composition further comprises the following components:

| | mg per unit dose of |
|---|---|
| Components | composition |
| Silicon Dioxide | 2.2 |
| Magnesium | 1.1 |
| Stearate | |

In another embodiment the composition comprises the following components:

| | mg per unit dose of |
|---|---|
| Components | composition |
| Formula (1.0A) | 30 |
| monohydrate | |
| Lactose Monohydrate | 86.02 |
| Microcrystalline Cellulose | 35.2 |
| Crospovidone | 16.5 |
| Povidone | 5.28 |
| Sodium Lauryl Sulfate | 3 |

In one embodiment, the composition further comprises the following components:

| | mg per unit dose of |
|---|---|
| Components | composition |
| Microcrystalline Cellulose | 24.2 |
| Crospovidone | 16.5 |

In one embodiment, the composition further comprises the following components:

| | mg per unit dose of |
|---|---|
| Components | composition |
| Silicon Dioxide | 2.2 |
| Magnesium | 1.1 |
| Stearate | |

In another embodiment the composition comprises the following components:

| | Mg per unit dose of |
|---|---|
| Components | composition |
| Formula (1.0A) | 1 |
| monohydrate | |
| Lactose Monohydrate | 5.64 |
| Microcrystalline | 2.8 |
| Cellulose | |
| Crospovidone | 2.8 |
| Poloxamer 188 | 1.2 |
| Povidone | 0.66 |
| Silicon Dioxide | 0.1 |

In one embodiment, the composition further comprises the following components:

| | Mg per unit dose of |
|---|---|
| Components | composition |
| Lactose Monohydrate | 50.6 |
| Microcrystalline | 120 |
| Cellulose | |
| Crospovidone | 12.2 |

In one embodiment, the composition further comprises the following components.

| | Mg per unit dose of |
|---|---|
| Components | composition |
| Silicon Dioxide | 2 |
| Magnesium Stearate | 1 |

In another embodiment the composition comprises the following components:

| | Mg per unit dose of |
|---|---|
| Components | composition |
| Formula (1.0A) | 10 |
| monohydrate | |
| Lactose Monohydrate | 56.4 |
| Microcrystalline | 28 |
| Cellulose | |
| Crospovidone | 28 |
| Poloxamer 188 | 12 |
| Povidone | 6.6 |
| Silicon Dioxide | 1 |

In one embodiment, the composition further comprises the following components:

| | Mg per unit dose of |
|---|---|
| Components | composition |
| Lactose Monohydrate | 58 |
| Microcrystalline Cellulose | 8 |
| Crospovidone | 10 |

In one embodiment, the composition further comprises the following components:

| | Mg per unit dose of |
|---|---|
| Components | composition |
| Silicon Dioxide | 1 |
| Magnesium Stearate | 1 |

The present invention also provides a composition comprising the following components:

| | Mg per unit dose of |
|---|---|
| Components | composition |
| Formula (1.0A) | 10 |
| monohydrate | |
| Lactose Monohydrate | 57.5 |
| Microcrystalline Cellulose | 28.8 |
| Crospovidone | 28 |
| Starch Pregelatinized | 12 |
| Poloxamer 188 | 12 |

In one embodiment, the composition further comprises the following components:

| | Mg per unit dose of |
|---|---|
| Components | composition |
| Microcrystalline Cellulose | 28.8 |
| Crospovidone | 8 |
| Starch Pregelatinized | 12 |

In one embodiment, the composition further comprises the following components:

| | Mg per unit dose of |
|---|---|
| Components | composition |
| Silicon Dioxide | 2 |
| Magnesium | 1 |
| Stearate | |

In another embodiment the composition comprises the following components:

| | Mg per unit dose of |
|---|---|
| Components | composition |
| Formula (1.0A) | 50 |
| monohydrate | |
| Lactose Monohydrate | 55.3 |
| Microcrystalline Cellulose | 25.1 |
| Crospovidone | 32.5 |
| Starch Pregelatinized | 15 |
| Poloxamer 188 | 15 |

In one embodiment, the composition further comprises the following components:

| | Mg per unit dose of |
|---|---|
| Components | composition |
| Microcrystalline Cellulose | 25.1 |
| Crospovidone | 12.5 |
| Starch Pregelatinized | 15 |

In one embodiment, the composition further comprises the following components:

| | Mg per unit dose of |
|---|---|
| Components | composition |
| Silicon Dioxide | 3 |
| Magnesium | 1.5 |
| Stearate | |

In one embodiment, the composition exhibits a mean AUC of the compound of formula (1.0A) between about 484 ng·hr/ml and about 489 ng·hr/ml following a single-dose oral administration of 30 mg of the compound of formula (1.0A) to a human. In one embodiment, the composition exhibits mean Cmax of the compound of formula (1.0A) between about 122 ng/ml and about 147 ng/ml following a single-dose oral administration of 30 mg of the compound of formula (1.0A) to a human. In one embodiment, the composition exhibits a median Tₘₐₓ of the compound of formula (1.0A) between about 0.5 and about 2 hours following oral administration to a human.

In one embodiment, the compositions of the present invention are for oral administration. For oral preparations, a pharmaceutically acceptable carrier (which includes diluents, excipients, or carrier materials) is also present in the composition. The carrier is suitably selected with respect to the intended form of administration, i.e., oral capsules (either solid-filled, semi-solid (gel) filled, or liquid filled), powders for constitution, oral gels, orally disintegrating tablet, orally consumable films, elixirs, syrups, suspensions, and the like, and consistent with conventional pharmaceutical practices. For example, for oral administration in the form of capsules, the pharmaceutically active agents may be combined with any oral non-toxic pharmaceutically acceptable inert carrier, such as lactose, starch, sucrose, cellulose, magnesium stearate, dicalcium phosphate, calcium sulfate, mannitol, ethyl alcohol (liquid forms), and the like. Moreover, when desired or needed, suitable binders, lubricants, disintegrants, disinfectants and coloring agents may also be incorporated in the mixture. Suitable binders include starch, gelatin, natural sugars, corn sweeteners, natural and synthetic gums such as acacia, sodium alginate, carboxymethylcellulose, polyethylene glycol, and waxes. Suitable lubricants include boric acid, sodium benzoate, sodium acetate, sodium chloride, and the like. Suitable disintegrants include starch, methylcellulose, guar gum, and the like. Suitable disinfectants include benzalkonium chloride and the like. Sweetening and flavoring agents and preservatives may also be included where appropriate.

Additionally, the compositions may be formulated in sustained release form to provide the rate controlled release of any one or more of the pharmaceutically active agents to optimize the therapeutic effects. Suitable compositions for sustained release include layered capsules (e.g., containing layers of varying disintegration rates or controlled release polymeric matrices impregnated with the medicinal agents) that are shaped in capsules containing such impregnated or encapsulated porous polymeric matrices.

In another embodiment, the compositions are for parenteral administration, for example, intravenous, intratumoral, subcutaneous, or intramuscular administration.

Thus, to prepare an aqueous solution for parenteral injection, it is possible to use a co-solvent, e.g., an alcohol such as ethanol or a glycol such as polyethylene glycol or propylene glycol, or glycerin, and optionally, a hydrophilic surfactant such as Tween® 80. An oily solution injectable intramuscularly can be prepared, e.g., by solubilizing the active principle with a triglyceride or a glycerol ester. The substantially non-aqueous carrier (excipient) can be any substance that is biocompatible and liquid or soft enough at body temperature. The carrier is usually hydrophobic and commonly organic, e.g., an oil or fat of vegetable, animal, mineral or synthetic origin or derivation. Preferably, but not necessarily, the carrier includes at least one chemical moiety of the kind that typifies “fatty” compounds, e.g., fatty acids, alcohols, esters, etc., i.e., a hydrocarbon chain, an ester linkage, or both. “Fatty” acids in this context include acetic, propionic and butyric acids, through straight- or branched-chain organic acids containing up to 30 or more carbon atoms.

Preferably, the carrier is immiscible in water and/or soluble in the substances commonly known as fat solvents. The carrier can correspond to a reaction product of such a “fatty” compound or compounds with a hydroxy compound, e.g., a mono-hydric, di-hydric, trihydric or other polyhydric alcohol, e.g., glycerol, propanediol, lauryl alcohol, polyethylene or -propylene glycol, etc. These compounds include the fat-soluble vitamins, e.g., tocopherols and their esters, e.g., acetates sometimes produced to stabilize tocopherols. Sometimes, for economic reasons, the carrier may preferably comprise a natural, unmodified vegetable oil such as sesame oil, soybean oil, peanut oil, palm oil, or an unmodified fat. Alternatively the vegetable oil or fat may be modified by hydrogenation or other chemical means which is compatible with the present invention. The appropriate use of hydrophobic substances prepared by synthetic means is also envisioned.

Pharmaceutical compositions suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g., human serum albumin), toxicity agents (e.g., NaCl), preservatives (e.g., thimerosol, cresol or benzylalcohol), and surfactants (e.g., Tween or polysorabates) in sterile water for injection.

Typical suitable syringes include systems comprising a prefilled vial attached to a pen-type syringe such as the NOVOLET Novo Pen available from Novo Nordisk, as well as prefilled, pen-type syringes which allow easy self-injection by the user. Other syringe systems include a pen-type syringe comprising a glass cartridge containing a diluent and lyophilized powder in a separate compartment.

Exemplary compositions of the compound of formula (1.0A) are given in Tables 1-4 below. Formulation 1 capsules containing the compound of formula (1.0) is detailed in Table 1.

TABLE 1

Amount per
capsule (mg)

| | 10 mg | 50 mg |
|---|---|---|
| | Formula | Formula |
| | (1.0A) | (1.0A) |
| Components | capsule | capsule |
| Formula (1.0A) | 10 | 50 |
| monohydrate | | |
| Lactose Monohydrate | 57.5 | 55.3 |
| Microcrystalline Cellulose | 28.8 | 25.1 |
| Crospovidone | 28 | 32.5 |
| Starch Pregelatinized | 12 | 15 |
| Poloxamer 188 | 12 | 15 |
| Purified Water USP | —^{a} | —^{a} |
| Total Granule Weight | 124.3 | 162.9 |
| Capsule Fill | | |
| Monohydrate Granules | 124.3 | 162.9 |
| Microcrystalline Cellulose | 28.8 | 25.1 |
| Crospovidone | 8 | 12.5 |
| Starch Pregelatinized | 12 | 15 |
| Silicon Dioxide | 2 | 3 |
| Magnesium Stearate | 1 | 1.5 |
| Capsule Fill Weight | 176 | 220 |
| Capsule Shell | | |
| Hard Gelatin Capsule, No. | 60 | — |
| 2 Blue Opaque^{b} | | |
| Hard Gelatin Capsule, No. | — | 75 |
| 1 Blue Opaque^{c} | | |
| Total Filled Capsule | 236 | 295 |
| Weight | | |

^{a}Evaporates during the manufacturing process
^{b}Contains 0.8867% FD&C Blue #2, 1.4393% Titanium Dioxide, and qs 100% gelatin.
^{c}Contains 0.8867% FD&C Blue #1, 1.4393% Titanium Dioxide, and qs 100% gelatin.

Formulation 1 capsules were manufactured via wet granulation using a low shear mixing process, drying, milling, blending, and encapsulation in hard gelatin capsules. These capsules were found to be stable for at least 6 months at 40° C./75% relative humidity (RH), and for at least 18 months at 25° C./60% RH when packaged in high density polyethylene bottles (HDPE) bottles.

Formulation 1, however, was not amenable to large scale processing due to the low-shear mixing process which is impractical for large scale processing. To facilitate a process scale-up using the wet granulation method, the low shear mixing process was replaced by a fluidized bed process. This change in manufacture however, also required a modification in the formulation as pregelatinized starch, the binder used in Formulation 1, is incompatible with the fluidized bed process adopted. Therefore, another binder compatible with both the fluidized bed process and the compound of formula (1.0A) was required. Povidone was subsequently identified as a suitable binder and employed in place of pregelatinized starch at an entirely different concentration. Formulation 2 containing the compound of formula (1.0A) as well as povidone is detailed in Table 2.

TABLE 2

Amount per
capsule (mg)

| | 1 mg | 10 mg |
|---|---|---|
| | Formual | Formual |
| | (1.0A) | (1.0A) |
| Components | capsule | capsule |
| Monohydrate Granules | | |
| Formual (1.0A) | 1 | 10 |
| monohydrate | | |
| Lactose Monohydrate | 5.64 | 56.4 |
| Microcrystalline Cellulose | 2.8 | 28 |
| Crospovidone | 2.8 | 28 |
| Poloxamer 188 | 1.2 | 12 |
| Povidone | 0.66 | 6.6 |
| Silicon Dioxide | 0.1 | 1 |
| Purified Water USP | —^{a} | —^{a} |
| Total Granule Weight | 14.2 | 142 |
| Capsule Fill | | |
| Monohydrate Granules | 14.2 | 142 |
| Lactose Monohydrate | 50.6 | 58 |
| Microcrystalline Cellulose | 120 | 8 |
| Crospovidone | 12.2 | 10 |
| Silicon Dioxide | 2 | 1 |
| Magnesium Stearate | 1 | 1 |
| Capsule Fill Weight | 200 | 220 |
| Capsule Shell | | |
| Hard Gelatin Capsule, No. 2 | 60 | 60 |
| Blue Opaque^{b} | | |
| Total Filled Capsule | 260 | 280 |
| Weight | | |

^{a}Evaporates during the manufacturing process
^{b}Contains 0.8867% FD&C Blue #2, 1.4393% Titanium Dioxide, and qs 100% gelatin.

Formulation 2 capsules were manufactured via wet granulation using a fluidized bed, drying, milling, blending, and encapsulation in hard gelatin capsules. Although amenable to large scale processing, Formulation 2 capsules were found to discolor during manufacture.

The formulations in Table 3 were prepared to provide formulations that are both amenable to large scale processing by wet granulation and that result in a more color stable product Exemplary formulations using poloxamer or SLS are provided in Table 3.

TABLE 3

Amount per capsule (mg)

| Components | Formulation A | Formulation B | |
|---|---|---|---|
| Polymorph Form III of | 30 | 30 | |
| formula (1.0A) | | | |
| monohydrate, | | | |
| micronized | | | |
| Lactose Monohydrate | 80.9 | 129 | |
| NF | | | |
| Cellulose | 13.4 | 20 | |
| Microcrystalline NF | | | |
| (Avicel PH 102) | | | |
| Crospovidone NF | 15.7 | 10 | |
| Povidone K29/32 | 7.4 | 8 | |
| Poloxamer 188 NF | 12.8 | — | |
| SLS | — | 3 | |
| Total | 160 | 200 | mg |

Based on the increased color stability of Formulation B which employs SLS as a wetting agent at 1.5% instead of poloxamer at either 3% or 8%, Formulation 3 containing the compound of formula (1.0A) and SLS was developed. Formulation 3 is detailed in Table 4.

TABLE 4

| | Amount per capsule (mg) | | |
|---|---|---|---|
| | 3 mg | 10 mg | 30 mg |
| | Compound | Compound | Compound |
| | 1.0A | 1.0A | 1.0A |
| Components | capsule | capsule | capsule |
| Monohydrate Granules | | | |
| Compound (1.0A) | 3 | 10 | 30 |
| monohydrate | | | |
| Lactose Monohydrate | 115.72 | 108.02 | 86.02 |
| Microcrystalline Cellulose | 35.2 | 35.2 | 35.2 |
| Crospovidone | 16.5 | 16.5 | 16.5 |
| Povidone | 5.28 | 5.28 | 5.28 |
| Sodium Lauryl Sulfate | 0.3 | 1 | 3 |
| Purified Water USP | —^{a} | —^{a} | —^{a} |
| Total Granule Weight | 176 | 176 | 176 |
| Capsule Fill | | | |
| Monohydrate Granules | 176 | 176 | 176 |
| Microcrystalline Cellulose | 24.2 | 24.2 | 24.2 |
| Crospovidone | 16.5 | 16.5 | 16.5 |
| Silicon Dioxide | 2.2 | 2.2 | 2.2 |
| Magnesium Stearate | 1.1 | 1.1 | 1.1 |
| Capsule Fill Weight | 220 | 220 | 220 |
| Capsule Shell | | | |
| Hard Gelatin Capsule, | 60 | 60 | 60 |
| No. 2 Blue Opaque^{b} | | | |
| Total Filled Capsule | 280 | 280 | 280 |
| Weight | | | |

^{a}Evaporates during the manufacturing process
^{b}Contains 0.8867% FD&C Blue #2, 1.4393% Titanium Dioxide, and qs 100% gelatin.

Formulation 3 capsules were manufactured in a manner similar to Formulation 2 via wet granulation using a fluidized bed, drying, milling, blending, and encapsulation.

Compound (1.0B) can be prepared by the examples described below.

### Example 1

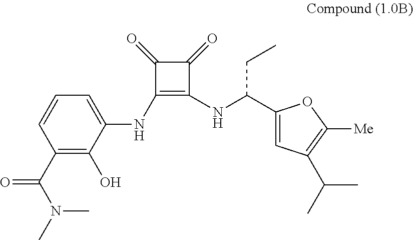

### Step 1

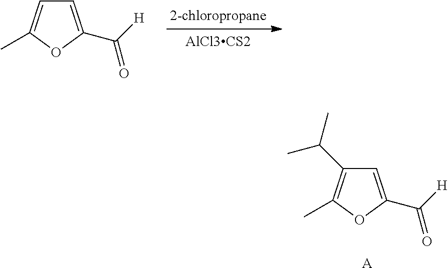

5-Methyl-furan-2-carbaldehyde (I) (2.0 moles) in CS₂ (300 ml) was added dropwise to the suspension of AlCl₃ (4 moles) in CS₂ (1.514 at 0° C. over 30 min. The reaction mixture was stirred at 0° C. for 15 min and at 10° C. for 1 h. The reaction mixture was carefully poured over ice-H₂O (10 L) and the aqueous layer was extracted with ether (3×4 L). The organic layer was washed with saturated NaHCO₃ (1.5 L), and H₂O (2.5 L). Dried over MgSO₄, filtered and concentrated under reduced pressure to yield a crude oil (275 g), which was purified by flash column chromatography with 0%-15% Ethyl acetate-Hexanes to provide compound (II) as a light-yellow oil 205 g (67%).

### Steps 2 to 6: Intermediates B to F

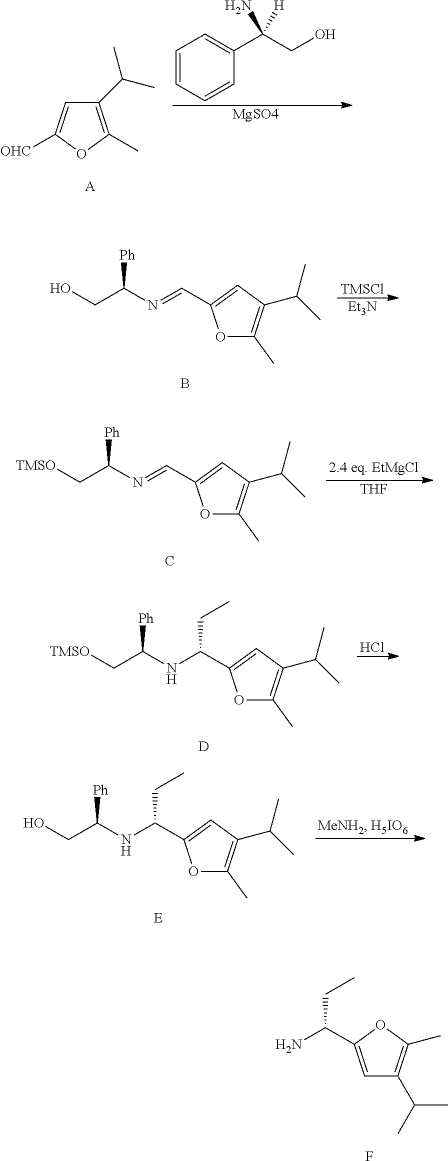

### Step 2: Preparation of Compound B

MgSO₄ (600 g) was added to a solution of Compound A (204 g, 1.314 mol) in dichloromethane (4 L) at room temperature. A solution of R-(−)-2-phenylglycinol (189.3 g, 1.38 mol) in dichloromethane (12 L) was added over 30 min. After 4 hours, MgSO₄ (200 g) was added. The mixture was stirred at room temperature over night. Solids were filtered and washed with dichloromethane (1 L). The filtrate was used directly in next reaction. ¹HNMR (CDCl₃): 8.04 (s, 1H), 7.41-7.26 (m, 5H), 6.67 (s, 1H), 4.39 (m, 1H), 4.03 (m, 1H), 3.88 (m, 1H), 2.77 (m, 1H), 2.31 (s, 3H), 1.14 (d, 6H).

### Step 3: Preparation of Compound C

Triethylamine (159.6 g, 1.58 mol) and dichloromethane (157.1 g, 1.45 mol) were added to above filtrate sequentially. The mixture was stirred at room temperature for 1 hour. Hexane (4 L) was added. Solids were filtered and washed with hexane. A reddish oil (464 g) was obtained upon concentration of the filtrate. ¹HNMR (CDCl₃): 8.02 (s, 1H), 7.45-7.24 (m, 5H), 6.64 (s, 1H), 4.31 (t, 1H), 3.90 (d, 2H), 2.76 (m, 1H), 2.30 (s, 3H), 1.14 (d, 6H).

### Steps 4 and 5: Preparation of Compound D and E

A solution of Compound C (454 g, 1.285 mol) in THF (1 L) was added slowly to a solution of 2M EtMgCl (1.56 L) in THF (2 L) at −35° C. It was stirred for 1 hour at −35° C. and then over night at room temperature to give Compound D.

HCl (4N, 1.8 L) was added slowly to the above mixture at 0° C. and stirred at room temperature for 3 hours. The reaction was diluted with diethylether (2 L) and hexane (3 L). The mixture was adjusted with NaOH (2N, ˜1 L) to pH ˜9. Organic layer was separated and washed with brine. Aqueous layer was acidified to pH 6 with HCl and extracted with EtOAc. All organic layers were combined and washed with brine. A sticky oil (Compound E, 401 g) was obtained upon concentration, ¹HNMR (CDCl₃): 7.28-7.17 (m, 5H), 6.86 (s, 11.4), 3.82 (m, 1H), 3.67 (m, 1H), 3.53 (m, 2H), 2.61 (m, 1H), 2.05 (s, 3H), 1.78 (m, 2H), 1.05 (d, 6H), 0.86 (t, 3H).

### Step 6: Preparation of Compound F

To a solution of Compound E (401 g) in MeOH (5.3 L), was added methylamine (40% water solution, 2.2 L), followed by a solution of periodic acid (898.4 g in 1.3 L water) between 25° C. to 35° C. It was stirred over night at room temperature. Solids were filtered and washed with MeOH (0.3 L) and diethylether (0.5 L). Diethylether (4 L), water (2 L) and brine (0.3 L) were added to the filtrate. More solid was precipitated out. Solids were filtered again and washed with MeOH and ether. More diethylether (2 L) and water (1 L) were added to the filtrate. Two layers were separated. Aqueous layer was extracted with diethylether (3 L). The combined diethylether layer was washed with brine.

HCl (3N, 1 L) was added to the above diethylether layer. It was stirred at room temperature for 30 min. Two layers were separated. Diethylether layer was washed with water (0.5 L). The combined aqueous layer was basified to pH 14 with 3N NaOH and extracted with diethylether twice (2×2 L). Diethylether layer was dried with Na₂SO₄ and concentrated to an oil (262 g). Oil was loaded on a filtration plug filled with 1.1 kg of silica gel. It was eluded with 50% to 100% of ethyl acetate (EA) in hexane and finally 2% of MeOH in EA. The combined filtrate was concentrated to give Compound F as a light brown oil (191 g). ¹HNMR (CDCl₃): 5.95 (s, 1H), 3.82 (m, 1H), 3.72 (t, 1H), 2.68 (m, 1H), 2.18 (s, 3H), 1.83-1.61 (m, 2H), 1.11 (d, 6H), 0.93 (t, 3H).

### Step 7

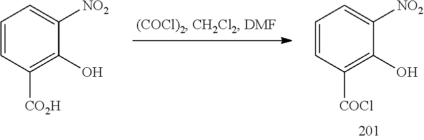

To a mixture of 3-nitrosalicylic acid (20 g, 0.109 mol), N,N-dimethylformamide (4 mL), and dichloromethane (500 mL) was added oxalyl chloride (18.6 mL, 0.225 mol, 2.06 eq) dropwise with stirring at room temperature. The reaction mixture was stirred at the same temperature for 2-3 h when all the solid in the mixture dissolved. Evaporation of solvent and excess oxalyl chloride and drying under vacuum afforded 22 g of the acid chloride 201 as a yellow solid, which was used in next step without purification.

### Step 8

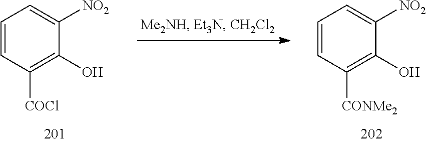

To a mixture of the acid chloride 201 (22 g, ca. 0.109 mol) and dichloromethane (400 mL) at 0° C. was added triethylamine (61 mL, 0.437 mol) slowly with stirring under argon, followed by slow addition of 2 M dimethylamine solution in tetrahydrofuran (108 mL, 0.218 mol). After addition, the mixture was stirred at room temperature overnight. The mixture was then concentrated under reduced pressure, and EtOAc (500 mL) and water (200 mL) were added. The organic layer was separated, washed with 1 N HCl solution, water, and brine, dried over Na₂SO₄, and concentrated under reduced pressure to give the amide 202 as a yellow solid (20.95 g, 91% over two steps). ¹H NMR (CDCl₃) δ 10.92 (s, 1H), 8.15 (d, 1H), 7.62 (d, 1H), 7.06 (t, 1H), 7.08 (d, 1H), 3.08 (s, 6H).

### Step 9

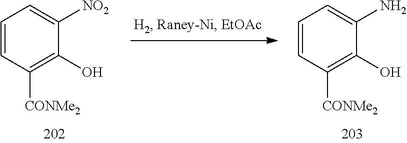

A mixture of the amide 202 (20.95 g, 99.7 mmol), EtOAc (200 mL), and Raney-nickel (3 spoons) was subjected to hydrogenation at 60 psi at room temperature overnight. The mixture was filtered through a layer of Celite. The filtrate was concentrated under reduced pressure to give the dark oil residue, which was purified by column chromatography (EtOAc-hexanes, 1:1) to give 11.13 g (62%) of the amine 203 as a colorless oil. ¹H NMR (CDCl₃) δ 6.80-6.65 (m, 3H) (d, 1H), 3.15 (s, 3H).

### Step 10

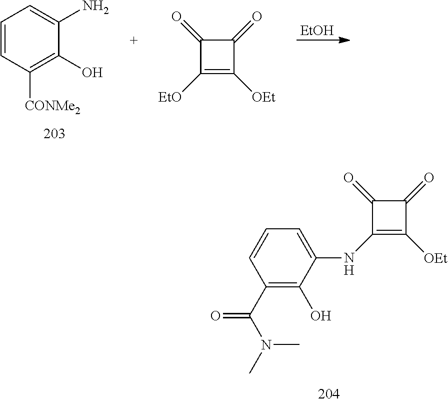

A mixture of the amine 203 (14.55 g, 80.74 mmol), EtOH (500 mL), and 3,4-diethoxy-3-cyclobutene-1,2-dione (14.4 g, 80.74 mmol) was stirred at room temperature overnight. The mixture was then concentrated under reduced pressure. The residue was purified by column chromatography (EtOAc-hexanes, 3:1) to give 20.46 g (84%) of the Compound 204 as a yellow solid. ¹H NMR (CDCl₃) δ 10.99 (s, 1H), 8.00-7.64 (m, 2H), 7.09 (d, 1H), 6.88 (t, 1H), 4.86 (q, 2H), 3.18 (s, 6H), 1.51 (t, 3H).

### Step 11

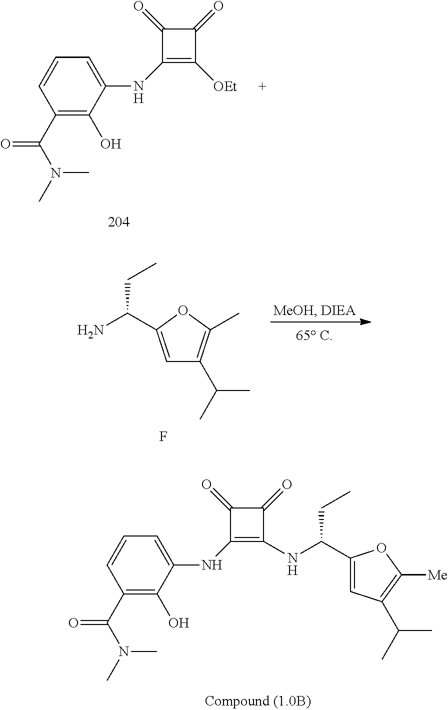

A mixture of the amine F (4.9 g, 27.07 mmol), the intermediate 204 (8 g, 26.32 mmol), diisopropylethylamine (0.6 mL), and ethanol (140 mL) was stirred at 65° C. overnight when TLC analysis (CH₂Cl₂-MeOH, 9:1) showed that the starting materials disappeared. The mixture was then concentrated under reduced pressure. The residue was purified by column chromatography (CH₂Cl₂-MeOH, 30:1) to give 8.2 g (71%) of the target Compound 1.08 as a pale brown solid. LC-MS: Rt 6.82 min, m/e 462.0, 900.9; ¹H NMR (DMSO-d₆) δ 9.85 (s, t H), 9.18 (s, 1H), 8.56 (d, 1H), 7.76 (d, 1H), 6.80 (m, 2H), 6.18 (s, 1H), 5.00 (m, 1H), 3.22 (s, 1H), 2.88 (s, 6H), 2.61 (m, 1H), 2.08 (s, 3H), 1.96-1.86 (m, 2H), 1.02 (d, 6H), 0.95 (t, 3H).

### Preparative Example 1

### Preparation of Compound (213) (HCl Salt of Compound (212))

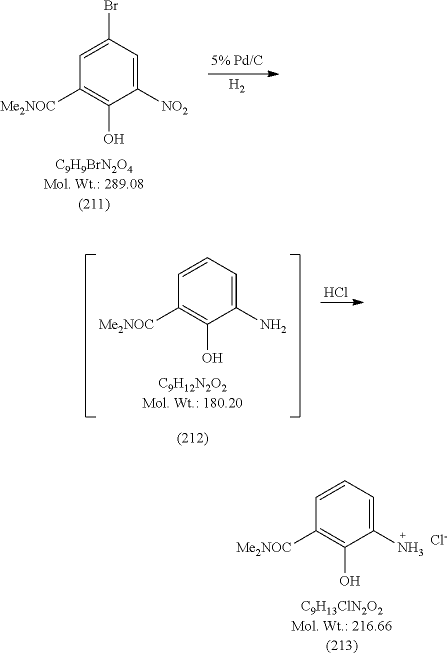

To a suspension of 10 g (34.6 mmol) of (211) in a mixture of 21 ml of methyl t-butylether and 49 ml of ethanol was added 13.7 ml of KOEt (24%) in ethanol, followed by addition of 0.8 g of 5% Pd/C (50% wet). The mixture was then agitated under 120-150 psi hydrogen pressure for about 6 hours. Upon completion of the reaction, the batch was filtered through a Celite pad and the cake was washed with 80 ml of solvent mixture of methyl t-butylether and ethanol (1:1). The filtrate was treated with 3.7 ml of concentrated HCl solution. The batch was then concentrated under reduced pressure to about 50 ml. Isopropanol (100 ml) was added and the resulting solution was concentrated under vacuum to about 40 mL Methyl t-butylether (50 ml) was added, followed by a slow addition of 110 ml of heptane. Finally, the mixture was cooled to 0° C. The solids were collected by filtration and the cake was washed with 20 ml solvent mixture of 1:1 methyl t-butylether/EtOH. The cake was dried at 60° C. for 10 hours in a vacuum oven, to give 7.24 g (96%) off-white solids. ¹H NMR (DMSO-D6): 7.50 (d, 1H), 6.96 (dd, 1H), 7.17 (d, 1H), 2.9 (br, 6H), 10.2 (br, 4H), ¹³C NMR (DMSO-D6): 147.7, 121.4, 125.9, 120.6, 128.5, 127.1, 167.8.

### Preparative Example 2

### Preparation of the Oxalate of Compound (212)

Following the procedure described for preparing the HCl salt (213) in Preparative Example 1, 10 g (34.6 mmol) of compound (211) was hydrogenated under the same condition and the filtered solution was treated with 3.3 g of oxalic acid. Following the same procedure as above resulted in 8.5 g (90%) off-white solids. ¹H NMR (DMSO-D6): 6.45 (m, 2H), 6.17 (dd, 1H), 2.70 (s, 6H), 5.5 (very broad, 4H).

### Preparative Example 3

### Preparation of the p-PTSA Salt of Compound (212)

Following the procedure described for preparing the HCl salt (213) in Preparative Example 1, 10 g of compound (211) was hydrogenated under the same condition and the filtrate was treated with 7.9 g (41.1 mmol) p-toluenesulfonic acid monohydrate. The resulting mixture was concentrated as above and the mixture after heptane addition was stirred over night at room temperature, to give 11.4 g (94%) off-white solids. ¹H NMR (DMSO-D6): 7.49 (d, 2H), 7.29 (d, 1H), 7.15 (m, 3H), 6.93 (dd, 1H), 2.90 (s, 6H), 2.31 (s, 3H).

### Preparative Example 4

### Preparation of Tartrate of Compound (212)

Following the procedure described for preparing the HCl salt (213) in Preparative Example 1, 10 g of compound (211) was hydrogenated under the same condition and the filtrate was treated with 5.47 g (36.5 mmol) of tartaric acid. Following the same procedure as described in 527123-PS preparation resulted in 9.1 g (80%) of off-white solids. ¹H NMR (DMSO-D6): 8.5 (br, 3H), 6.6 (dd, 2H), 6.38 (d, 1H), 4.26 (s, 2H), 3.6 (b, 2H), 2.96 (s, 6H).

### Preparative Example 5

### Preparation of Compound 209A

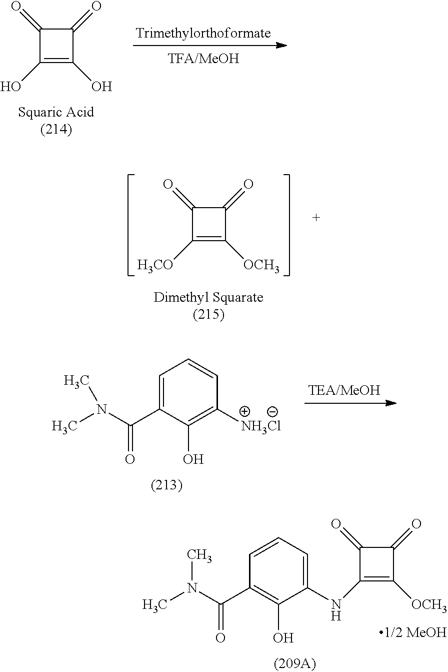

Charged 9.5 kg of the compound of formula 214 to 50 gallon glass reactor equipped with a thermocouple, N₂ inlet and feed tank. Charged 65 liters dry methanol (KF<0.1%) followed by 20 liters trimethylorthoformate and 0.2 kg trifluororacetic acid. Heated the batch to reflux and maintained for about one hour. Concentrated the batch at one atmosphere until the internal temperature exceeded 70° C. Maintained the batch at reflux for about four hours. Adjusted the batch to a temperature between 40 and 50° C. and charged 26 liters dry methanol. Adjusted the temperature to about 20 to 30° C. Charged 78 liters of dry methanol and adjusted the batch to a temperature between −5 and 5° C. Charged 13.0 kg of the compound of formula V. Over about 4 hours, charged 11.1 kg triethylamine (TEA) to the batch while maintaining the batch at a temperature between −5 and 5° C. About one and a half hours after the start of the TEA charge, seeded the batch with 130 grams of compound (209A) added as a solid. After the addition of TEA was completed, agitated the batch for about 30 minutes at a temperature between −5 and 5° C. Charged 12 liters acetic acid while maintaining the batch at a temperature between −5 and 5° C. Heated the batch to a temperature between 60 and 70° C. and maintained this temperature for about 1 hour. Adjusted the temperature to about 25 to 35° C. over about 1 hour. Adjusted the temperature to about −5 to 5° C. over about 1 hour. Filtered the batch and washed the filter cake with 65 liters (5×) methanol. Dried the batch in a vacuum oven for at least 24 hours at 60 to 70° C. Yield 14.5 kg, 81%. ¹HNMR (CD₃CN) 8.07 (1H, s); 7.56 (1H, d); 7.28 (1H, d); 6.99 (1H, t); 4.35 (3H, s); 3.10 (6H, s)

### Preparative Example 6

### Preparation of Compound (209A) from Dimethylsquarate and Compound (213)

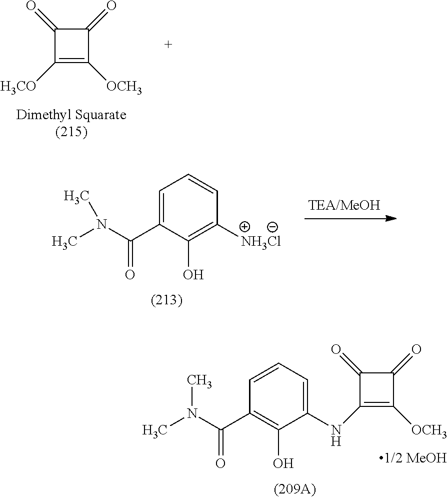

Charged 6.3 grams of compound (213) and 5.0 grams of compound (215) to 250 ml round bottom flask equipped with a thermocouple, N₂ inlet and addition funnel. Charged 41 ml dry methanol (KF<0.1%). Adjusted the batch to temperature between −5 and 5° C. Over about 5 hours, charged 4.9 ml triethylamine (TEA) to the batch while maintaining the batch at a temperature between −5 and 5° C. After the addition of TEA was completed, agitated the batch for about one hour at a temperature between −5 and 5° C. Charged 2.8 ml acetic acid while maintaining the batch at a temperature between −5 and 5° C. Adjusted the batch volume to 63 ml by adding dry methanol. Heated the batch to reflux and maintained for about 15 minutes. Adjusted the temperature to about −5 to 5° C. over about 1 hour. Filtered the batch and washed the filter cake with 25 ml (5×) methanol. Dried the batch in a vacuum oven for at least 24 hours at 60 to 70° C. Yield 7.5 g, 88%.

### Preparative Example 7

### Preparation of Compound (209B) from Diethylsquarate (216) and Compound (213)

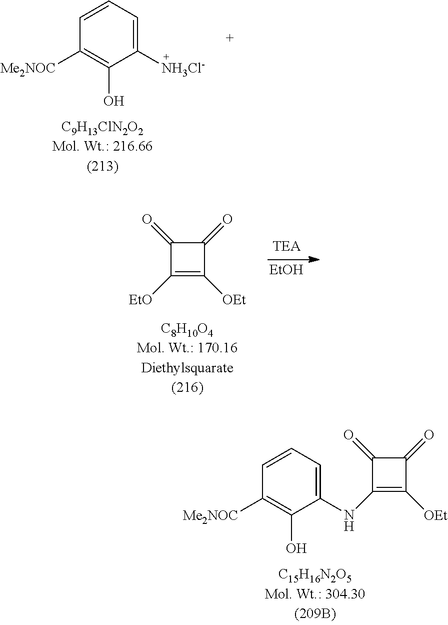

Charged 44.0 kg of the compound (213), 225 kg dry ethanol and 41.8 kg of the compound (216) to a 300 gallon glass lined reactor equipped with a thermocouple, N₂ inlet and feed bottle. Adjusted the batch to temperature between 0 and 10° C. Over about 1 hour, charged 17.1 kg triethylamine (TEA) to the batch while maintaining the batch at a temperature between 0 and 10° C. After the addition of TEA was complete, agitated the batch for about three hours at a temperature between 0 and 10° C. Over about 3 hours, charged additional 8.2 kg triethylamine (TEA) to the batch while maintaining the batch at a temperature between 0 and 10° C. After the addition of TEA was complete, agitated the batch for about three hours at a temperature between 0 and 10° C. Charged 19 liters acetic acid while maintaining the batch at a temperature between 0 and 10° C. Adjusted the batch volume to 440 liters by adding dry ethanol. Heated the batch to reflux and maintained for about 15 minutes. Adjusted the temperature to about 0 to 10° C. over about 2 hours. Filtered the batch and washed the filter cake with 220 liters 50% v/v ethanol in water. Dried the batch in a vacuum oven for at least 12 hours at 50 to 60° C. Yield 52 kg, 88%.

¹HNMR (CD₃CN) 7.61 (1H, d); 7.28 (1H, d); 6.96 (1H, t); 4.69 (2H, q); 3.10 (6H, s), 1.44 (3H, t).

### Example 2

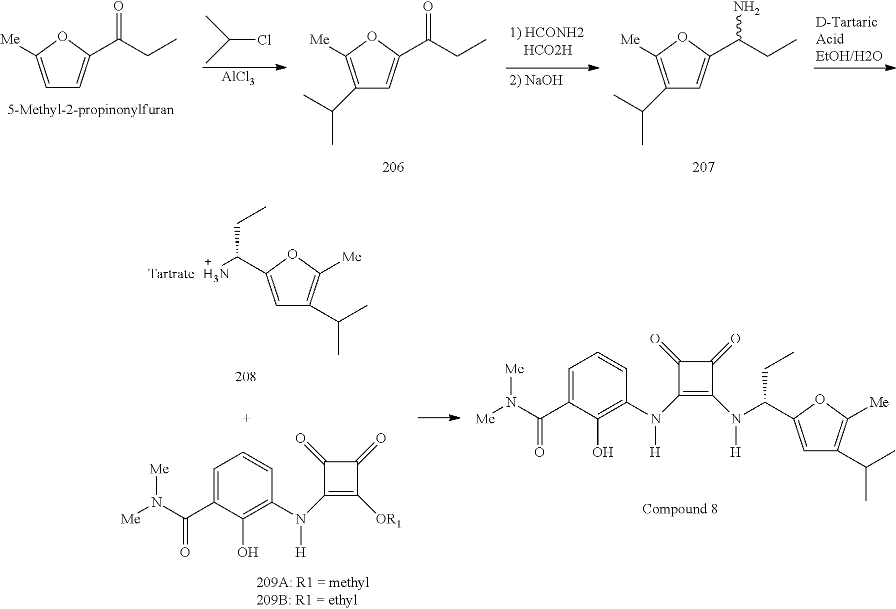

### Step 1 1-(4-Isopropyl-5-methyl-2-furyl)propan-1-one (206)

Under nitrogen, 2-methyl-5-propionylfurane (100 g, 0.72 moles) was added dropwise at 0-30° C. to aluminium chloride (131 g, 0.96 moles). The resulting suspension was stirred for further 30 minutes at room temperature and then cooled to 0-5° C. Within one hour isopropyl chloride (76 g, 0.96 moles) was added dropwise at 0-10° C. and the mixture stirred until complete conversion was achieved (HPLC). The mixture was hydrolyzed on 2 L of water/ice. The pH was adjusted to 1 by addition of sodium hydroxide solution (60 mL) and the product was extracted into 500 mL TBME. The aqueous layer was separated and reextracted with 200 mL TBME. The combined organic layers were washed with 500 mL brine and evaporated to minimum volume. Yield: 132.5 g (102%) of a yellow-brown liquid.

Assay (HPLC: YMC Pack Pro C18 150×4.6 mm, 5 μm; 220 nm; ACN/0.05% TFA:water/0.05% TFA 20:80 to 95:5 within 23 min): 60% pure by area, RT 17.2 min.

### Step 2 [1-(4-Isopropyl-5-methyl-2-furyl)propyl]amine (207)

Under nitrogen, a mixture of crude 1-(4-Isopropyl-5-methyl-2-furyl)propan-1-one (100 g), formamide (100 g, 2.22 moles) and formic acid (28.7 g, 0.61 moles) was heated to 140° C. for about two days until complete conversion to intermediate N-(1-(4-isopropyl-5-methylfuran-2-yl)propyl)formamide was achieved. The mixture was cooled to 20-25° C. and diluted with 400 mL methanol and 400 mL diisopropylether. Aqueous sodium hydroxide (1.2 kg, 25% in water) was added and the mixture was heated to reflux (55-60° C.) for about one day until complete conversion to [1-(4-Isopropyl-5-methyl-2-furyl)propyl]amine was achieved. The mixture was cooled down to 20-25° C. and the phases were separated. The organic layer was washed with 400 mL brine (5% in water). The combined aqueous layers were reextracted with 200 mL diisopropylether. The combined organic layers were evaporated to minimum volume. Yield: 94.6 g (45% abs (absolute), from 2-methyl-5-propionylfurane) of a yellow-brown liquid.

Assay (HPLC: YMC Pack Pro C18 150×4.6 mm, 5 μm; 220 nm; ACN/0.05% TFA:water/0.05% TFA 20:80 to 95:5 within 23 min): 48.5% pure vs. standard, RT 9.2 min.

### Step 3 (R)-1-(4-Isopropyl-5-methylfuran-2-yl)propan-1-amine (2S,3S)-2,3-dihydroxysuccinate (208)

Under nitrogen, crude [1-(4-isopropyl-5-methyl-2-furyl)propyl]amine (51 g, 135 mmol active) was dissolved in 204 mL dry ethanol at 60° C. 20% of a solution of D-(−)-tartaric acid (20.3 g, 135 mmol) in a mixture of 102 mL ethanol/water (15:1) was added at 55° C. The solution was seeded. The residual solution of tartaric acid was added within 10 minutes. The suspension was cooled to 20° C. and stirred at room temperature over night. The salt was filtered off and washed with dry ethanol until a colorless mother liquor was obtained. The product was dried in vacuum at 50° C. to constant weight. Yield: 16.9 g (38% abs.) of white crystals.

Assay (HPLC: YMC Pack Pro 018 150×4.6 mm, 5 μm; 220 nm; ACN:0.01M KH₂PO₄ pH=2.5 (H₃PO₄) 15:85 to 80:20 within 25 min): 95.8% by area, RT 8.8 min.

Optical Purity (HPLC: Chiralcel OD-R 250×4.6 mm; 226 nm; ACN:0.5M NaClO₄ 40:60): dr 98:2, RT 12.6 min (R), 16.3 min (S). Wherein “dr” represents diastereomeric ratio.

### Step 4 2-Hydroxy-3-[(2-{[(1R)-1-(4-isopropyl-5-methyl-2-furyl)propyl]amino}-3,4-dioxocyclobut-1-en-1-yl)amino]-N,N-dimethylbenzamide (Compound (1.0B))

Under nitrogen, (R)-1-(4-Isopropyl-5-methylfuran-2-yl)propan-1-amine (2S,3S)-2,3-dihydroxy-succinate (208)(2.0 g, 6 mmol) was suspended in 6 ml water and 8 mL 2-methyl tetrahydrofurane (MeTHF) at 20-25° C. 1.3 mL aqueous sodium hydroxide (30%) were added and the organic layer was separated after 5 minutes. The aqueous layer was extracted with 4 ml. MeTHF. The combined organic layers were added to (209B) (1.74 g, 5.7 mmol) and 4 ml. MeTHF were added. The mixture was heated to 65° C. for 4.5 hours and was then cooled to 20-25° C. After 16 hours at 20-25° C. the product crystallized and was isolated by filtration. The product was washed with MeTHF and dried in vacuum at 50° C. to constant weight. Yield: 1.25 g (47%) as off-white solid. Assay (NMR): 95% pure.

If one were to use compound (209A) in Example 3 then one would obtain compound (1.0B).

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

1. A method of treating chronic obstructive disease in a patient in need of such treatment, said method comprising administering to said patient an effective amount of a CXCR2 antagonist and administering an effective amount of at least one drug selected from the group consisting of: angiotensin-converting enzyme inhibitors, Angiotensin II receptor antagonists, cardioselective beta blockers, and lipid regulating drugs.

2. The method of claim 1 wherein said CXCR2 antagonist is selected from the group consisting of: 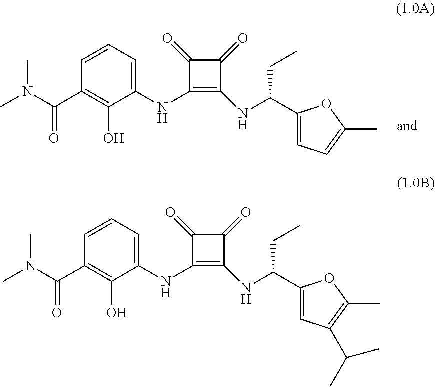
or a pharmaceutically acceptable salt, ester, solvate, or polymorph thereof.

3. The method of claim 1 wherein said CXCR2 antagonist is: 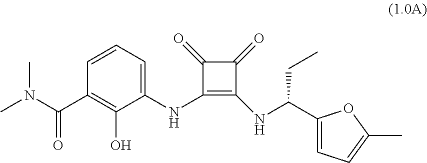
or a monohydrate thereof, or a polymorph thereof.

4. (canceled)

5. The method of claim 1 wherein:
(A) said angiotensin-converting enzyme inhibitors are selected from the group consisting of: (a) Benazepril HCl, (b) Captopril, (c) Moexipril hydrochloride, (d) Perindopril erbumine, (e) Lisinopril, (f) Ramipril, and (g) Trandolapril;
(B) said Angiotensin II receptor antagonists are selected from the group consisting of: (a) Eprosartan mesylate, (b) Irbesartan, (c) Losartan potassium, (d) Olmesartan medoxomil, (e) Telmisartan, (f) Valsartan, and (g) Candesartan Cilexetil;
(C) said cardioselective beta blockers are selected from the group consisting of: (a) Metoprolol succinate, and (b) Metoprolol tartrate; and
(D) said lipid regulating drugs are selected from the group consisting of: (a) Atorvastatin calcium, (b) Fluvastatin sodium, (c) Lovastatin, (d) Rosuvastatin calcium, (e) Simvastatin, and (f) Ezetimibe in combination with Simvastatin.

6-8. (canceled)

9. The method of claim 1 wherein said CXCR2 antagonist is: 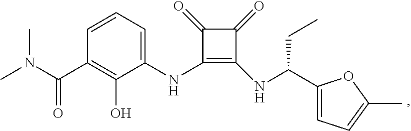
or a monohydrate thereof, or a polymorph thereof, and
(A) said angiotensin-converting enzyme inhibitors are selected from the
group consisting of: (a) Benazepril HCl, (b) Captopril, (c) Moexipril hydrochloride, (d) Perindopril erbumine, (e) Lisinopril (f) Ramipril, and (g) Trandolapril,
(B) said Angiotensin II receptor antagonists are selected from the group consisting of: (a) Eprosartan mesylate, (b) Irbesartan, (c) Losartan potassium, (d) Olmesartan medoxomil, (e) Telmisartan, (f) Valsartan, and (g) Candesartan Cilexetil,
(C) said cardioselective beta blockers are selected from the group consisting of: (a) Metoprolol succinate, and (b) Metoprolol tartrate, and
(D) said lipid regulating drugs are selected from the group consisting of: (a) Atorvastatin calcium, (b) Fluvastatin sodium (c) Lovastatin, (d) Rosuvastatin calcium, (e) Simvastatin, and (f) Ezetimibe in combination with Simvastatin.

10. The method of claim 1 wherein said CXCR2 antagonist is: 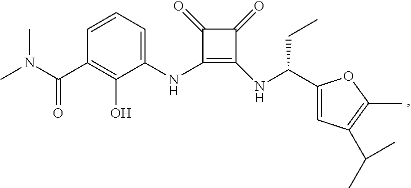
and
(A) said angiotensin-converting enzyme inhibitors are selected from the group consisting of: (a) Benazepril HCl, (b) Captopril, (c) Moexipril hydrochloride, (d) Perindopril erbumine, (e) Lisinopril (f) Ramipril, and (g) Trandolapril,
(B) said Angiotensin II receptor antagonists are selected from the group consisting of: (a) Eprosartan mesylate, (b) Irbesartan, (c) Losartan potassium, (d) Olmesartan medoxomil, (e) Telmisartan, (f) Valsartan, and (g) Candesartan Cilexetil,
(C) said cardioselective beta blockers are selected from the group consisting of: (a) Metoprolol succinate, and (b) Metoprolol tartrate, and
(D) said lipid regulating drugs are selected from the group consisting of: (a) Atorvastatin calcium, (b) Fluvastatin sodium (c) Lovastatin, (d) Rosuvastatin calcium, (e) Simvastatin, and (f) Ezetimibe in combination with Simvastatin.

11. The method of claim 9 wherein said CXCR2 antagonist is administered in combination at least one lipid regulating drug.

12. The method of claim 11 wherein said lipid regulating drug is Simvastatin.

13-14. (canceled)

15. The method of claim 1 wherein
(A) said angiotensin-converting enzyme inhibitors are selected from the group consisting of:
(a) Benazepril HCl administered in amounts of 5 to 40 mg per day,
(b) Captopril administered in amounts of 25 to 300 mg per day,
(c) Moexipril hydrochloride administered in amounts of 7.5 to 30 mg daily,
(d) Perindopril erbumine administered in amounts of 2 to 8 mg a day,
(e) Lisinopril administered in amounts of 10 to 40 mg per day,
(f) Ramipril administered in amounts of 2.5 to 20 mg once daily,
(g) Trandolapril administered in amounts of 1 to 4 mg daily, and
(B) said Angiotensin II receptor antagonists are selected from the group consisting of:
(a) Eprosartan mesylate administered for a total amount of 400 to 800 mg a day,
(b) Irbesartan administered in amounts of 75 to 300 mg daily,
(c) Losartan potassium administered in a total amount of about 25 to 100 mg daily,
(d) Olmesartan nnedoxomil administered in amounts of 20 to 40 mg once daily,
(e) Telmisartan administered in amounts of 20 to 80 mg once daily, and
(f) Valsartan administered in amounts of 80 to 320 mg once per day, and
(C) said cardioselective beta blockers are selected from the group consisting of:
(a) Metoprolol succinate administered in amounts of 25 to 100 mg daily, and
(d) Metoprolol tartrate administered in amounts of 100 to 450 mg daily, and
(D) said lipid regulating drugs are selected from the group consisting of:
(a) Atorvastatin calcium administered in amounts of 10 to 80 mg once daily,
(b) Fluvastatin sodium administered in amounts of 20 to 80 mg per day,
(c) Lovastatin administered in amounts of 10 to 80 mg per day,
(d) Rosuvastatin calcium administered in amounts of 5 to 40 mg once daily,
(e) Simvastatin administered in amounts of 5 to 40 mg a day, and
(f) Ezetimibe in combination with Simvastatin administered in amounts of 10 mg per day of Ezetimibe, and 10 to 80 mg per day of Simvastatin.

16. The method of claim 15 wherein the CXCR2 antagonist is 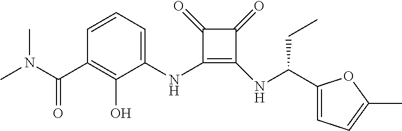
or a monohydrate thereof, or a polymorph thereof.

17. The method of claim 16 wherein said CXCR2 antagonist is administered in doses of 3 to 30 mg once a day.

18-19. (canceled)

20. A method of treating Chronic Obstructive Pulmonary Disease in a patient in need of such treatment, said method comprising administering to said patient 3 to 30 mg per day of the CXCR2 antagonist 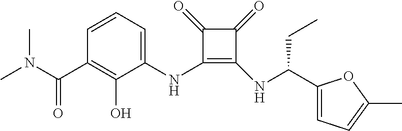
or a monohydrate thereof, or a polymorph thereof, in combination with Simvastatin administered in a dose of 5 to 40 mg once a day,

21. A method of treating Chronic Obstructive Pulmonary Disease in a patient in need of such treatment, said method comprising administering to said patient 3 to 30 mg per day of the CXCR2 antagonist 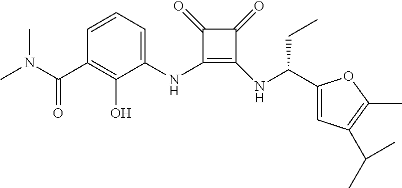
in combination with Simvastatin administered in a dose of 5 to 40 mg once a day.

22. A pharmaceutical composition comprising:
(A) an effective amount of at least one CXCR2 antagonist and an effective amount of at least one drug selected from the group consisting of: angiotensin-converting enzyme inhibitors, Angiotensin II receptor antagonists, cardioselective beta blockers, and lipid regulating drugs; or
(B) an effective amount of 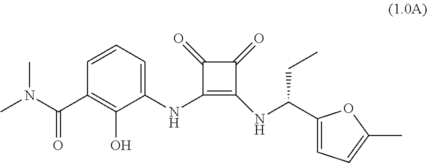
or a monohydrate thereof, or a polymorph thereof, and a pharmaceutically acceptable carrier, and an effective amount of at least one drug selected from the group consisting of: angiotensin-converting enzyme inhibitors, Angiotensin II receptor antagonists, cardioselective beta blockers, and lipid regulating drugs; or
(C) an effective amount of 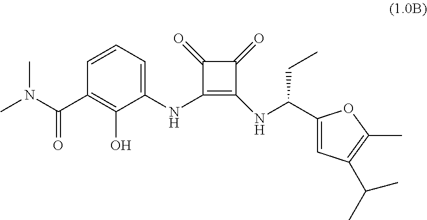
and a pharmaceutically acceptable carrier, and an effective amount of at least one drug selected from the group consisting of: angiotensin-converting enzyme inhibitors, Angiotensin II receptor antagonists, cardioselective beta blockers, and lipid regulating drugs.

23-24. (canceled)
